(19) United States
(12) Patent Application Publication
(10) Pub. No.: US 2009/0247743 A1
(43) Pub. Date: Oct. 1, 2009

(54) PREPARATION OF BETA-AMINO ACIDS HAVING AFFINITY FOR THE ALPHA-2-DELTA PROTEIN

(75) Inventors: Brian G Conway, Gales Ferry, CT (US); Thomas Norman Nanninga, Kalamazoo, MI (US); Hiafeng Wu, Hackettstown, NJ (US); Garrett S. Hoge, Basel (CH); Bruce Allen Pearlman, Westfield, NJ (US); Derick Dale Winkle, Holland, MI (US)

Correspondence Address: PFIZER INC;Mary J Hosley, 150 EAST 42ND STREET, MS: 150/02/E112, NEW YORK, NY, 10017-5612, US

(73) Assignee: Pfizer Inc

(21) Appl. No.: 11/909,318

(22) PCT Filed: Jun. 27, 2005
(86) PCT No.: PCT/IB05/01966

§ 371 Date: Sep. 21, 2007

Related U.S. Application Data
(60) Provisional application No. 60/586,512, filed on Jul. 9, 2004.
(60) Provisional application No. 60/665,041, filed on Mar. 24, 2005.

(51) Int. Cl. C07D 205/08; C07C 227/16; C07C 67/313; C07C 69/533

(52) U.S. Cl. 540/200; 562/553; 560/129; 560/205

(57) ABSTRACT

Disclosed are materials and methods for preparing optically active β-amino acids of Formula 1,

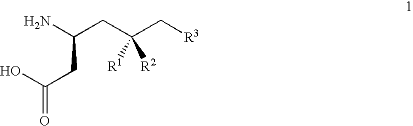

which bind to the alpha-2-delta (α2δ) subunit of a calcium channel.

### BACKGROUND OF THE INVENTION

1. Field of Invention

This invention relates to materials and methods for preparing optically-active β-amino acids that bind to the alpha-2-delta (α2δ) subunit of a calcium channel. These compounds, including their pharmaceutically acceptable complexes, salts, solvates and hydrates, are useful for treating pain, fibromyalgia, and a variety of psychiatric and sleep disorders.

2. Discussion

U.S. Patent Application No. 2003/0195251 A1 to Barta et al. (the '251 application) describes β-amino acids that bind to the α2δ subunit of a calcium channel. These compounds, including their pharmaceutically acceptable complexes, salts, solvates, and hydrates, may be used to treat a number of disorders, conditions, and diseases. These include, without limitation, sleep disorders, such as insomnia; fibromyalgia; epilepsy; neuropathic pain, including acute and chronic pain; migraine; hot flashes; pain associated with irritable bowel syndrome; restless leg syndrome; anorexia; panic disorder; depression; seasonal affective disorders; and anxiety, including general anxiety disorder, obsessive compulsive behavior, and attention deficit hyperactivity disorder, among others.

Many of the β-amino acids described in the '251 application are optically active. Some of the compounds, like those represented by Formula 1 below, possess two or more stereogenic (chiral) centers, which make their preparation challenging. Although the '251 application describes useful methods for preparing optically-active β-amino acids at laboratory bench scale, many of the methods may be problematic for pilot- or full-scale production because of efficiency or cost concerns. Thus, improved methods for preparing optically-active β-amino acids, such as those given by Formula 1, would be desirable.

### SUMMARY OF THE INVENTION

The present invention provides comparatively efficient and cost-effective methods for preparing compounds of Formula 1,

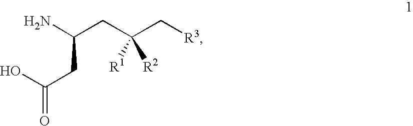

or a diastereomer thereof or a pharmaceutically acceptable complex, salt, solvate or hydrate thereof, wherein:

R¹ and R² are independently hydrogen atoms or C₁₋₃ alkyl optionally substituted with one to five fluorine atoms, provided that when R¹ is a hydrogen atom, R² is not a hydrogen atom; and

R³ is C₁₋₄ alkyl, C₃₋₄ cycloalkyl, C₃₋₆ cycloalkyl-C₁₋₆ alkyl, aryl, aryl-C₁₋₃ alkyl, or arylamino, wherein each alkyl of R³ is optionally substituted with one to five fluorine atoms, and each aryl of R³ is optionally substituted with from one to three substituents independently selected from chloro, fluoro, amino, nitro, cyano, C₁₋₃ alkylamino, C₁₋₃ alkyl optionally substituted with one to three fluorine atoms, and C₁₋₃ alkoxy optionally substituted with from one to three fluorine atoms.

One aspect of the present invention includes reacting a compound of Formula 2,

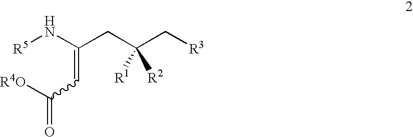

### or Formula 4,

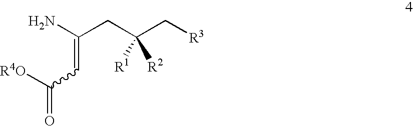

with H₂ in the presence of a chiral catalyst to give a compound of Formula 3,

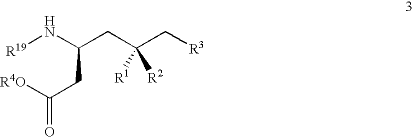

or a diastereomer thereof, wherein

R¹, R², and R³ in Formula 2, Formula 3, and Formula 4 are as defined in Formula 1;

R⁴ in Formula 2, Formula 3, and Formula 4 is a hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₄ alkyl, aryl-C₂₋₄ alkenyl, or aryl-C₂₋₄ alkynyl or a cation selected from a Group 1 metal ion, a Group 2 metal ion, a primary ammonium ion or a secondary ammonium ion; and

R⁵ in Formula 2 and R¹⁹ in Formula 3 are independently hydrogen atom, carboxy, C₁₋₇ alkanoyl, C₂₋₇ alkenoyl, C₂₋₇ alkynoyl, C₃₋₇ cycloalkanoyl, C₃₋₇ cycloalkenoyl, halo-C₁₋₇ alkanoyl, halo-C₂₋₇ alkenoyl, halo-C₂₋₇ alkynoyl, C₁₋₆ alkoxycarbonyl, halo-C₁₋₆ alkoxycarbonyl, C₃₋₇ cycloalkoxycarbonyl, aryl-C₁₋₇ alkanoyl, aryl-C₂₋₇ alkenoyl, aryl-C₂₋₇ alkynoyl, aryloxycarbonyl, or aryl-C₁₋₆ alkoxycarbonyl, provided that R⁵ is not a hydrogen atom; and

optionally converting the compound of Formula 3 or its diastereomer to the compound of Formula 1 or its diastereomer or to a pharmaceutically acceptable complex, salt, solvate or hydrate of the compound of Formula 1 or its diastereomer.

A useful chiral catalyst for asymmetric hydrogenation of the compounds of Formula 2 or Formula 4 includes a chiral ligand bound to a transition metal through one or more phosphorus atoms. Such catalysts include (R,R,S,S)-TANGPhos, (R)-BINAPINE, (R)-eTCFP, or (R)-mTCFP, or stereoisomers thereof, which are bound to rhodium. The asymmetric hydrogenation is typically carried out using a single chiral catalyst. However, the method may also employ multiple chiral catalysts in which the prochiral substrate (Formula 2 or Formula 4) is reacted successively with first and second chiral catalysts (e.g., (R)-BINAPINE and (R)-mTCFP, respectively, or opposite enantiomers thereof). In such cases, the first chiral catalyst has greater stereoselectivity than the second chiral catalyst under the same conditions, and the second chiral catalyst has a faster rate of reaction than the first chiral catalyst under the same conditions.

The compound of Formula 2 may be prepared by reacting the compound of Formula 4,

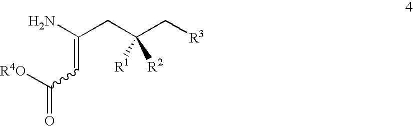

or a salt thereof with a compound of Formula 5,

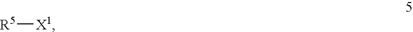

wherein R⁵ in Formula 5 is as defined in Formula 2 and X¹ in Formula 5 is a hydroxy or a leaving group, such as halogeno, aryloxy or heteroaryloxy, or —OC(O)R¹⁵, in which R¹⁵ is C₁₋₆ alkyl, C₂₋₆ alkenyl, C₂₋₆ alkynyl, C₃₋₁₂ cycloalkyl, halo-C₁₋₆ alkyl, halo-C₂₋₆ alkenyl, halo-C₂₋₆ alkynyl, aryl, aryl-C₁₋₆ alkyl, heterocyclyl, heteroaryl, or heteroaryl-C₁₋₆ alkyl.

The compound of Formula 4 may be prepared by reacting a compound of Formula 6,

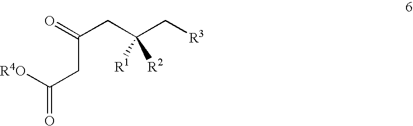

with an ammonia source (e.g., ammonia or a mixture of ammonium acetate and acetic acid), wherein R¹, R², and R³ in Formula 6 are as defined in Formula 1 and R⁴ is as defined in Formula 2.

Another aspect of the present invention includes reducing an amino moiety of a compound of Formula 7,

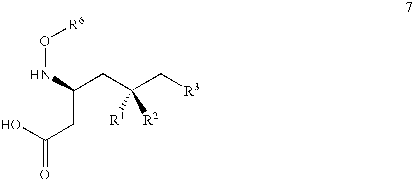

or a diastereomer thereof or a salt thereof to give the compound of Formula 1, wherein R¹, R², and R³ in Formula 7 are as defined in Formula 1 and R⁶ is C₁₋₆ alkyl (e.g., methyl), C₂₋₆ alkenyl (e.g., allyl) or aryl-C₁₋₃ alkyl (e.g., benzyl); and

optionally converting the compound of Formula 1 or its diastereomer to a pharmaceutically acceptable complex, salt, solvate or hydrate.

The amino moiety may be reduced by reacting the compound of Formula 7 with H₂ in the presence of a catalyst. Useful catalysts include transition metal catalysts, such as Pd/C and Raney nickel.

The compound of Formula 7 may be prepared by reacting a compound of Formula 8,

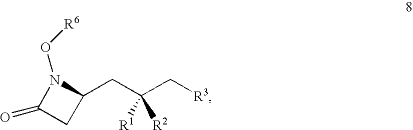

or a diastereomer thereof, with an acid or base, wherein R¹, R², and R³ in Formula 8 are as defined in Formula 1 and R⁶ is as defined in Formula 7.

The compound of Formula 8 may be prepared by cyclizing a compound of Formula 9,

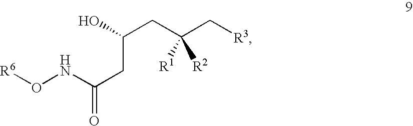

or a diastereomer thereof, wherein R¹, R², and R³ in Formula 9 are as defined in Formula 1 and R⁶ is as defined in Formula 7. For instance, the hydroxy moiety in Formula 9 may be activated (e.g., by conversion to a sulfonate ester) to give an activated alcohol, which is subsequently cyclized by treatment with a base (e.g., a carbonate).

The compound of Formula 9 may be prepared by reacting a compound of Formula 10,

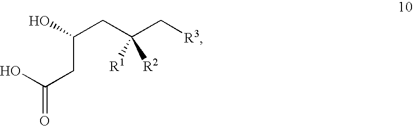

or a diastereomer thereof with a compound of Formula 11,

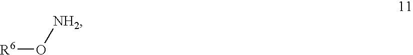

wherein R¹, R², and R³ in Formula 10 are as defined in Formula 1 and R⁶ in Formula 11 is as defined in Formula 7. To facilitate reaction, the carboxylic acid moiety of the compound of Formula 10 may be activated using a coupling agent, such as DMT-MM.

The compound of Formula 10 may be prepared by reacting the above compound of Formula 6 with H₂ in the presence of a chiral catalyst to give a compound of Formula 12,

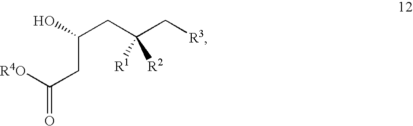

or a diastereomer thereof, wherein R¹, R², R³, and R⁴ in Formula 12 are as defined in Formula 1 and Formula 2; and

optionally converting the compound of Formula 12, or its diastereomer, to the compound of Formula 10.

An additional aspect of the present invention includes reducing an amine moiety of a compound of Formula 13,

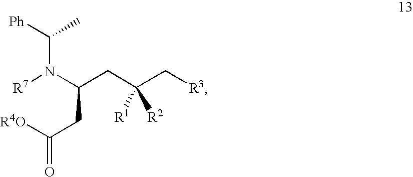

or a diastereomer thereof, to give a compound of Formula 37,

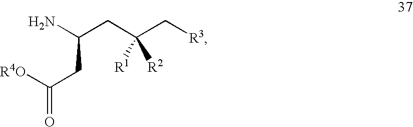

or a diastereomer thereof, wherein R¹, R², R³, and R⁴ in Formula 37 and Formula 13 are as defined in Formula 1 and Formula 2, respectively, and R⁷ in Formula 13 is C₁₋₆ alkyl, C₂₋₆ alkenyl (e.g., allyl) or aryl-C₁₋₃ alkyl (e.g., benzyl); and

optionally converting the compound of Formula 37 or its diastereomer to the compound of Formula 1 or its diastereomer or to a pharmaceutically acceptable complex, salt, solvate or hydrate of the compound of Formula 1 or its diastereomer.

The amine moiety of the compound of Formula 13 may be reduced by reacting the compound of Formula 13 with H₂ in the presence of a catalyst. Useful catalysts include transition metal catalysts, such as Pd/C and Raney nickel.

The compound of Formula 13 may be prepared by treating a compound of Formula 14,

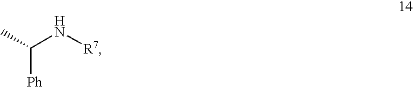

or its opposite enantiomer with a base to give a deprotonated chiral amine and reacting the deprotonated chiral amine with a compound of Formula 15,

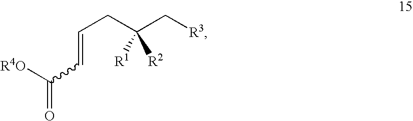

wherein R¹, R², and R³ in Formula 15 are as defined in Formula 1, R⁴ in Formula 15 is as defined in Formula 2, and R⁷ in Formula 14 is as defined in Formula 13.

The compound of Formula 15 may be prepared by reacting a compound of Formula 16,

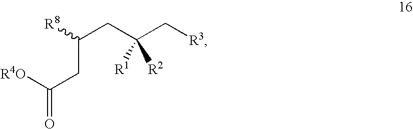

with a base, wherein R¹, R², and R³ in Formula 16 are as defined in Formula 1, R⁴ in Formula 16 is as defined in Formula 2, and R⁸ is a leaving group.

The compound of Formula 16 may be prepared by reacting a compound of Formula 17,

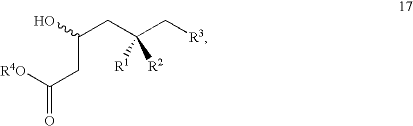

with a compound of Formula 18,

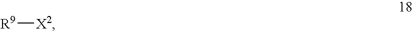

to give the compound of Formula 16 in which R⁸ is R⁹⁰—, wherein R¹, R², and R³ in Formula 17 are as defined in Formula 1, R⁴ in Formula 17 is as defined in Formula 2, R⁹ is tosyl, mesyl, brosyl, closyl, nosyl, or triflyl, and X² is halogen or R⁹O—.

The compound of Formula 17 may be prepared by reducing a β-carbonyl moiety of the above compound of Formula 6. For example, the compound of Formula 6 may be reacted with H₂ in the presence of a catalyst to give the compound of Formula 17. Useful catalysts include transition metal catalysts, such as platinum and ruthenium-based catalysts.

Alternatively, the compound of Formula 15 may be prepared by reacting a compound of Formula 39,

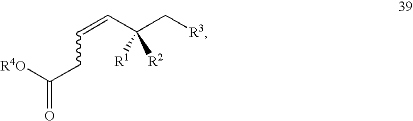

with a base, wherein R¹, R², and R³ in Formula 39 are as defined in Formula 1 and R⁴ in Formula 39 is as defined in Formula 2.

The compound of Formula 39 may be prepared by reacting a compound of Formula 38,

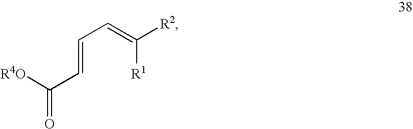

with a compound of Formula 29,

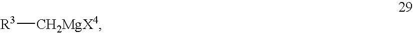

in the presence of copper and a chiral catalyst, wherein R¹, R², and R³ in Formula 29 and 38 are as defined in Formula 1, R⁴ in Formula 38 is as defined in Formula 2, and X⁴ in Formula 29 is halogeno.

The present invention also provides methods of making compounds of Formula 6, above. Thus, another aspect of the present invention includes treating a compound of Formula 19,

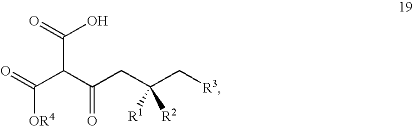

or a salt thereof with an acid, wherein R¹, R², R³, and R⁴ in Formula 19 are as defined in Formula 1 and Formula 2.

The compound of Formula 19 may be prepared by reacting a compound of Formula 20,

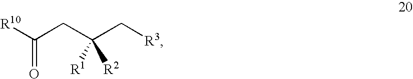

with a compound of Formula 21,

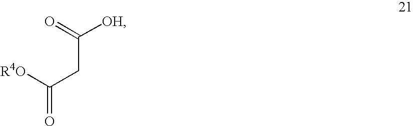

or a salt thereof, in the presence of a base and, optionally, a metal ion, wherein R¹, R², and R³ in Formula 20 and R⁴ in Formula 21 are as defined in Formula 1 and Formula 2, and R¹⁰ in Formula 20 is a leaving group, such as a chiral oxazolidin-2-one-3-yl or an imidazol-1-yl. Useful chiral oxazolidin-2-one-3-yls include (S)-4-isopropyloxazolidin-2-one-3-yl, (R)-4-isopropyloxazolidin-2-one-3-yl, (S)-4-benzyloxazolidin-2-one-3-yl, (R) -4-benzyloxazolidin-2-one-3-yl, (S)-4-phenyloxazolidin-2-one-3-yl, (R)-4-phenyloxazolidin-2-one-3-yl, (4S,5R)-4-methyl-5-phenyloxazolidin-2-one-3-yl, or (4R,5S)-4-methyl-5-phenyloxazolidin-2-one-3-yl or stereoisomers thereof.

The compound of Formula 20 may be prepared by reacting a compound of Formula 22,

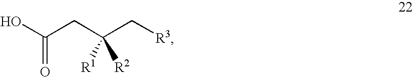

or a salt thereof, with coupling agent. Useful coupling agents include CDI, DCC, DMT-MM, FDPP, TATU, BOP, PyBOP, EDCI, diisopropyl carbodiimide, isopropenyl chloroformate, isobutyl chloroformate, N,N-bis-(2-oxo-3-oxazolidinyl)-phosphinic chloride, diphenylphosphoryl azide, diphenylphosphinic chloride, or diphenylphosphoryl cyanide.

The compound of Formula 22 may be prepared by hydrolyzing a compound of Formula 23,

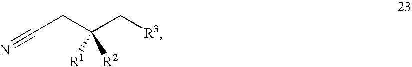

in the presence of an acid, wherein R¹, R², and R³ in Formula 23 are as defined in Formula 1.

The compound of Formula 23 may be prepared by reacting a compound of Formula 24,

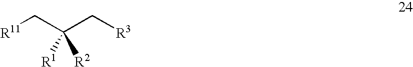

with a source of cyanide ion, wherein R¹, R², and R³ in Formula 24 are as defined in Formula 1 and R¹¹ is a leaving group. Useful sources of cyanide ion include sodium cyanide, potassium cyanide, zinc cyanide, hydrogen cyanide or acetone cyanohydrin, alone or in combination.

The compound of Formula 24 may be prepared by reacting a compound of Formula 25,

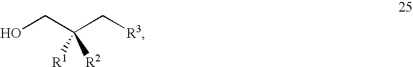

with a compound of Formula 26,

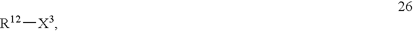

to give the compound of Formula 24 in which R¹¹ is R¹²O—, wherein R¹, R², and R³ in Formula 25 are as defined in Formula 1, R¹² in Formula 26 is a tosyl, mesyl, brosyl, closyl, nosyl, or triflyl, and X³ is halogen.

Alternatively, the compound of Formula 22 may be prepared by hydrolyzing a compound of Formula 27,

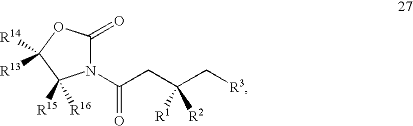

wherein R¹, R², and R³ in Formula 27 are as defined in Formula 1, and R¹³, R¹⁴, R¹⁵, and R¹⁶ are independently hydrogen atom, C₁₋₆ alkyl, C₃₋₆ cycloalkyl, C₃₋₆ cycloalkyl-C₁₋₆ alkyl, aryl, or aryl-C₁₋₃ alkyl, provided that R¹⁵ and R¹⁶ are different and are not both hydrogen atoms.

An additional aspect of the present invention includes treating a compound of Formula 33,

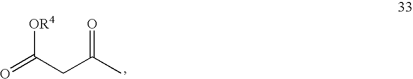

with a base to generate a dianion;

reacting the dianion with a compound of Formula 32,

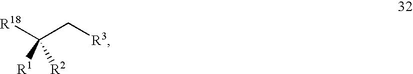

to give an intermediate; and

treating the intermediate with an acid to give the compound of Formula 6, wherein R¹, R², and R³ in Formula 32 and R⁴ in Formula 33 are as defined in Formula 1 and Formula 2, and R¹⁸ in Formula 32 is a leaving group.

The compound of Formula 32 may be prepared by reacting a compound of Formula 34,

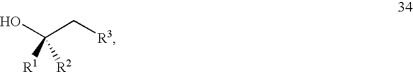

with the compound of Formula 26, above, to give the compound of Formula 32 in which R¹⁸ is R¹²O—, wherein R¹, R², and R³ in Formula 34 are as defined in Formula 1.

The present invention also provides compounds represented by Formula 2 to 4, 6 to 10, 12, 13, 15 to 17, 19, 20, 22, and 39, which are given above, and includes their complexes, salts, solvates, hydrates, opposite enantiomers, diastereomers, geometric isomers, and mixtures.

Thus, another aspect of the present invention provides compounds of Formula 40,

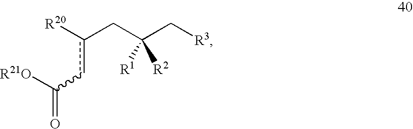

including complexes, salts, solvates, hydrates, opposite enantiomers, diastereomers, geometric isomers, and mixtures thereof, in which:

R¹, R², and R³ in Formula 40 are as defined above in Formula 1;

R²⁰ is a hydrogen atom, hydroxy, R⁶—O—NH—, R⁹⁰— or R¹⁹—NH—, or

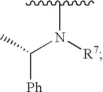

R²¹ is a hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl or a cation selected from a Group 1 metal ion, a Group 2 metal ion, a primary ammonium ion, a secondary ammonium ion or R⁶—O—NH—; and

R⁶, R⁷, R⁹, and R¹⁹ are as defined in Formula 7, Formula 13, Formula 18, and Formula 3, respectively.

A further aspect of the present invention provides compounds of Formula 39,

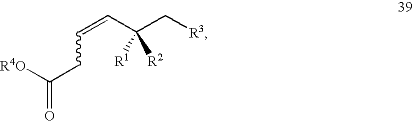

including complexes, salts, solvates, hydrates, opposite enantiomers, diastereomers, geometric isomers, and mixtures thereof, in which R¹, R², and R³ in Formula 39 are as defined in Formula 1, and R⁴ is as defined above in Formula 2.

An additional aspect of the present invention provides compounds of Formula 41,

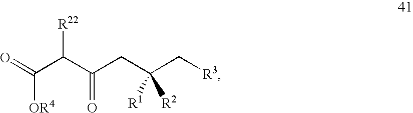

including complexes, salts, solvates, hydrates, opposite enantiomers, diastereomers, geometric isomers, and mixtures thereof, in which R¹, R², and R³ in Formula 41 are as defined in Formula 1, R⁴ is as defined in Formula 2, and R²² is a hydrogen atom or carboxy.

Yet another aspect of the present invention provides compounds of Formula 42,

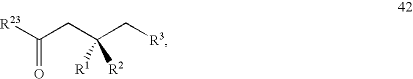

including complexes, salts, solvates, hydrates, opposite enantiomers, diastereomers, geometric isomers, and mixtures thereof, in which R¹, R², and R³ in Formula 42 are as defined in Formula 1, and R²³ is a hydrogen atom or a chiral oxazolidin-2-one-3-yl.

The present invention also includes the following compounds, as well as their pharmaceutically acceptable complexes, salts, solvates, hydrates, opposite enantiomers, diastereomers, geometric isomers, and mixtures:
(R)-5-methyl-3-oxo-heptanoic acid ethyl ester;
(R)-5-methyl-3-oxo-octanoic acid ethyl ester;
(R)-5-methyl-3-oxo-nonanoic acid ethyl ester;
(R,Z)-3-amino-5-methyl-hept-2-enoic acid ethyl ester;
(R,Z)-3-amino-5-methyl-oct-2-enoic acid ethyl ester;
(R,Z)-3-amino-5-methyl-non-2-enoic acid ethyl ester;
(R,Z)-3-acetylamino-5-methyl-hept-2-enoic acid ethyl ester;
(R,Z)-3-acetylamino-5-methyl-oct-2-enoic acid ethyl ester;
(R,Z)-3-acetylamino-5-methyl-non-2-enoic acid ethyl ester;
(3S,5R)-3-amino-5-methyl-heptanoic acid ethyl ester;
(3S,5R)-3-amino-5-methyl-octanoic acid ethyl ester;
(3S,5R)-3-amino-5-methyl-nonanoic acid ethyl ester;
(3S,5R)-3-acetylamino-5-methyl-heptanoic acid ethyl ester;
(3S,5R)-3-acetylamino-5-methyl-octanoic acid ethyl ester;
(3S,5R)-3-acetylamino-5-methyl-nonanoic acid ethyl ester;
(3S,5R)-3-acetylamino-5-methyl-heptanoic acid;
(3S,5R)-3-acetylamino-5-methyl-octanoic acid;
(3S,5R)-3-acetylamino-5-methyl-nonanoic acid;
(3R,5R)-3-hydroxy-5-methyl-heptanoic acid;
(3R,5R)-3-hydroxy-5-methyl-octanoic acid;
(3R,5R)-3-hydroxy-5-methyl-nonanoic acid;
(3R,5R)-3-hydroxy-5-methyl-heptanoic acid benzyloxy-amide;
(3R,5R)-3-hydroxy-5-methyl-octanoic acid benzyloxy-amide;
(3R,5R)-3-hydroxy-5-methyl-nonanoic acid benzyloxy-amide;
(3R,5R)-3-hydroxy-5-methyl-heptanoic acid ethyl ester;
(3R,5R)-3-hydroxy-5-methyl-octanoic acid ethyl ester;
(3R,5R)-3-hydroxy-5-methyl-nonanoic acid ethyl ester;
(2R,4S)-1-benzyloxy-4-(2-methyl-butyl)-azetidin-2-one;
(2R,4S)-1-benzyloxy-4-(2-methyl-pentyl)-azetidin-2-one;
(2R,4S)-1-benzyloxy-4-(2-methyl-hexyl)-azetidin-2-one;
(3S,5R)-3-benzyloxyamino-5-methyl-heptanoic acid;
(3S,5R)-3-benzyloxyamino-5-methyl-octanoic acid;
(3S,5R)-3-benzyloxyamino-5-methyl-nonanoic acid;
(1S,3S,5R)-3-[benzyl-(1-phenyl-ethyl)-amino]-5-methyl-heptanoic acid ethyl ester;
(1S,3S,5R)-3-[benzyl-(1-phenyl-ethyl)-amino]-5-methyl-octanoic acid ethyl ester;
(1S,3S,5R)-3-[benzyl-(1-phenyl-ethyl)-amino]-5-methyl-nonanoic acid ethyl ester;
(5R)-3-hydroxy-5-methyl-heptanoic acid ethyl ester;
(5R)-3-hydroxy-5-methyl-octanoic acid ethyl ester;
(5R)-3-hydroxy-5-methyl-nonanoic acid ethyl ester;
(R,E)-5-methyl-hept-2-enoic acid ethyl ester;
(R,E)-5-methyl-oct-2-enoic acid ethyl ester;
(R,E)-5-methyl-non-2-enoic acid ethyl ester;
(R,E)-5-methyl-hept-3-enoic acid ethyl ester;
(R,E)-5-methyl-oct-3-enoic acid ethyl ester;
(R,E)-5-methyl-non-3-enoic acid ethyl ester;
(5R)-5-methyl-3-(toluene-4-sulfonyloxy)-heptanoic acid ethyl ester;
(5R)-5-methyl-3-(toluene-4-sulfonyloxy)-octanoic acid ethyl ester;
(5R)-5-methyl-3-(toluene-4-sulfonyloxy)-nonanoic acid ethyl ester;
(5R)-3-methanesulfonyloxy-5-methyl-heptanoic acid ethyl ester;
(5R)-3-methanesulfonyloxy-5-methyl-octanoic acid ethyl ester;
(5R)-3-methanesulfonyloxy-5-methyl-nonanoic acid ethyl ester;
(R)-1-imidazol-1-yl-3-methyl-pentan-1-one;
(R)-1-imidazol-1-yl-3-methyl-hexan-1-one; and
(R)-1-imidazol-1-yl-3-methyl-heptan-1-one.

The present invention includes all complexes and salts, whether pharmaceutically acceptable or not, solvates, hydrates, and polymorphic forms of the disclosed compounds. Certain compounds may contain an alkenyl or cyclic group, so that cis/trans (or Z/E) stereoisomers are possible, or may contain a keto or oxime group, so that tautomerism may occur. In such cases, the present invention generally includes all Z/E isomers and tautomeric forms, whether they are pure, substantially pure, or mixtures.

### DETAILED DESCRIPTION

### Definitions and Abbreviations

Unless otherwise indicated, this disclosure uses definitions provided below. Some of the definitions and formulae may include a dash (“—”) to indicate a bond between atoms or a point of attachment to a named or unnamed atom or group of atoms. Other definitions and formulae may include an equal sign (“=”) or an identity symbol (“≡”) to indicate a double bond or a triple bond, respectively. Certain formulae may also include one or more asterisks (“*”) to indicate stereogenic (asymmetric or chiral) centers, although the absence of an asterisk does not indicate that the compound lacks a stereocenter. Such formulae may refer to the racemate or to individual enantiomers or to individual diastereomers, which may or may not be pure or substantially pure. Other formulae may include one or more wavy bonds [symbol] When attached to a stereogenic center, the wavy bonds refer to both stereoisomers, either individually or as mixtures. Likewise, when attached to a double bond, the wavy bonds indicate a Z-isomer, an E-isomer, or a mixture of Z and E isomers. Some formulae may include a dashed bond “ [symbol] ” to indicate a single or a double bond.

“Substituted” groups are those in which one or more hydrogen atoms have been replaced with one or more non-hydrogen atoms or groups, provided that valence requirements are met and that a chemically stable compound results from the substitution.

“About” or “approximately,” when used in connection with a measurable numerical variable, refers to the indicated value of the variable and to all values of the variable that are within the experimental error of the indicated value (e.g., within the 95% confidence interval for the mean) or within ±10 percent of the indicated value, whichever is greater.

“Alkyl” refers to straight chain and branched saturated hydrocarbon groups, generally having a specified number of carbon atoms (i.e., C₁₋₆ alkyl refers to an alkyl group having 1, 2, 3, 4, 5, or 6 carbon atoms). Examples of alkyl groups include, without limitation, methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkenyl” refers to straight chain and branched hydrocarbon groups having one or more unsaturated carbon-carbon bonds, and generally having a specified number of carbon atoms. Examples of alkenyl groups include, without limitation, ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms. Examples of alkynyl groups include, without limitation, ethynyl, 1-propyn-1-yl, 2-propyn-1-yl, 1-butyn-1-yl, 3-butyn-1-yl, 3-butyn-2-yl, 2-butyn-1-yl, and the like.

“Alkanoyl” refers to alkyl-C(O)—, where alkyl is defined above, and generally includes a specified number of carbon atoms, including the carbonyl carbon. Examples of alkanoyl groups include, without limitation, formyl, acetyl, propionyl, butyryl, pentanoyl, hexanoyl, and the like.

“Alkenoyl” and “alkynoyl” refer, respectively, to alkenyl-C(O)— and alkynyl-C(O)—, where alkenyl and alkynyl are defined above. References to alkenoyl and alkynoyl generally include a specified number of carbon atoms, excluding the carbonyl carbon. Examples of alkenoyl groups include, without limitation, propenoyl, 2-methylpropenoyl, 2-butenoyl, 3-butenoyl, 2-methyl-2-butenoyl, 2-methyl-3-butenoyl, 3-methyl-3-butenoyl, 2-pentenoyl, 3-pentenoyl, 4-pentenoyl, and the like. Examples of alkynoyl groups include, without limitation, propynoyl, 2-butynoyl, 3-butynoyl, 2-pentynoyl, 3-pentynoyl, 4-pentynoyl, and the like.

“Alkoxy” and “alkoxycarbonyl” refer, respectively, to alkyl-O—, alkenyl-O, and alkynyl-O, and to alkyl-O—C(O)—, alkenyl-O—C(O)—, alkynyl-O—C(O)—, where alkyl, alkenyl, and alkynyl are defined above. Examples of alkoxy groups include, without limitation, methoxy, ethoxy, n-propoxy, i-propoxy, n-butoxy, s-butoxy, t-butoxy, n-pentoxy, s-pentoxy, and the like. Examples of alkoxycarbonyl groups include, without limitation, methoxycarbonyl, ethoxycarbonyl, n-propoxycarbonyl, i-propoxycarbonyl, n-butoxycarbonyl, s-butoxycarbonyl, t-butoxycarbonyl, n-pentoxycarbonyl, s-pentoxycarbonyl, and the like.

“Halo,” “halogen” and “halogeno” may be used interchangeably, and refer to fluoro, chloro, bromo, and iodo.

“Haloalkyl,” “haloalkenyl,” “haloalkynyl,” “haloalkanoyl,” “haloalkenoyl,” “haloalkynoyl,” “haloalkoxy,” and “haloalkoxycarbonyl” refer, respectively, to alkyl, alkenyl, alkynyl, alkanoyl, alkenoyl, alkynoyl, alkoxy, and alkoxycarbonyl groups substituted with one or more halogen atoms, where alkyl, alkenyl, alkynyl, alkanoyl, alkenoyl, alkynoyl, alkoxy, and alkoxycarbonyl are defined above. Examples of haloalkyl groups include, without limitation, trifluoromethyl, trichloromethyl, pentafluoroethyl, pentachloroethyl, and the like.

“Cycloalkyl” refers to saturated monocyclic and bicyclic hydrocarbon rings, generally having a specified number of carbon atoms that comprise the ring (i.e., C₃₋₇ cycloalkyl refers to a cycloalkyl group having 3, 4, 5, 6 or 7 carbon atoms as ring members). The cycloalkyl may be attached to a parent group or to a substrate at any ring atom, unless such attachment would violate valence requirements. Likewise, the cycloalkyl groups may include one or more non-hydrogen substituents unless such substitution would violate valence requirements. Useful substituents include, without limitation, alkyl, alkenyl, alkynyl, haloalkyl, haloalkenyl, haloalkynyl, alkoxy, alkoxycarbonyl, alkanoyl, and halo, as defined above, and hydroxy, mercapto, nitro, and amino.

Examples of monocyclic cycloalkyl groups include, without limitation, cyclopropyl, cyclobutyl, cyclopentyl, cyclohexyl, and the like. Examples of bicyclic cycloalkyl groups include, without limitation, bicyclo[1.1.0]butyl, bicyclo[1.1.1]pentyl, bicyclo[2.1.0]pentyl, bicyclo[2.1.1]hexyl, bicyclo[3.1.0]hexyl, bicyclo[2.2.1]heptyl, bicyclo[3.2.0]heptyl, bicyclo[3.1.1]heptyl, bicyclo[4.1.0]heptyl, bicyclo[2.2.2]octyl, bicyclo[3.2.1]octyl, bicyclo[4.1.1]octyl, bicyclo[3.3.0]octyl, bicyclo[4.2.0]octyl, bicyclo[3.3.1]nonyl, bicyclo[4.2.1]nonyl, bicyclo[4.3.0]nonyl, bicyclo[3.3.2]decyl, bicyclo[4.2.2]decyl, bicyclo[4.3.1]decyl, bicyclo[4.4.0]decyl, bicyclo[3.3.3]undecyl, bicyclo[4.3.2]undecyl, bicyclo[4.3.3]dodecyl, and the like.

“Cycloalkenyl” refers monocyclic and bicyclic hydrocarbon rings having one or more unsaturated carbon-carbon bonds and generally having a specified number of carbon atoms that comprise the ring (i.e., C₃₋₇ cycloalkenyl refers to a cycloalkenyl group having 3, 4, 5, 6 or 7 carbon atoms as ring members). The cycloalkenyl may be attached to a parent group or to a substrate at any ring atom, unless such attachment would violate valence requirements. Likewise, the cycloalkenyl groups may include one or more non-hydrogen substituents unless such substitution would violate valence requirements. Useful substituents include, without limitation, alkyl, alkenyl, alkynyl, haloalkyl, haloalkenyl, haloalkynyl, alkoxy, alkoxycarbonyl, alkanoyl, and halo, as defined above, and hydroxy, mercapto, nitro, and amino.

“Cycloalkanoyl” and “cycloalkenoyl” refer to cycloalkyl-C(O)— and cycloalkenyl-C(O)—, respectively, where cycloalkyl and cycloalkenyl are defined above. References to cycloalkanoyl and cycloalkenoyl generally include a specified number of carbon atoms, excluding the carbonyl carbon. Examples of cycloalkanoyl groups include, without limitation, cyclopropanoyl, cyclobutanoyl, cyclopentanoyl, cyclohexanoyl, cycloheptanoyl, 1-cyclobutenoyl, 2-cyclobutenoyl, 1-cyclopentenoyl, 2-cyclopentenoyl, 3-cyclopentenoyl, 1-cyclohexenoyl, 2-cyclohexenoyl, 3-cyclohexenoyl, and the like.

“Cycloalkoxy” and “cycloalkoxycarbonyl” refer, respectively, to cycloalkyl-O— and cycloalkenyl-O and to cycloalkyl-O—C(O)— and cycloalkenyl-O—C(O)—, where cycloalkyl and cycloalkenyl are defined above. References to cycloalkoxy and cycloalkoxycarbonyl generally include a specified number of carbon atoms, excluding the carbonyl carbon. Examples of cycloalkoxy groups include, without limitation, cyclopropoxy, cyclobutoxy, cyclopentoxy, cyclohexoxy, 1-cyclobutenoxy, 2-cyclobutenoxy, 1-cyclopentenoxy, 2-cyclopentenoxy, 3-cyclopentenoxy, 1-cyclohexenoxy, 2-cyclohexenoxy, 3-cyclohexenoxy, and the like. Examples of cycloalkoxycarbonyl groups include, without limitation, cyclopropoxycarbonyl, cyclobutoxycarbonyl, cyclopentoxycarbonyl, cyclohexoxycarbonyl, 1-cyclobutenoxycarbonyl, 2-cyclobutenoxycarbonyl, 1-cyclopentenoxycarbonyl, 2-cyclopentenoxycarbonyl, 3-cyclopentenoxycarbonyl, 1-cyclohexenoxycarbonyl, 2-cyclohexenoxycarbonyl, 3-cyclohexenoxycarbonyl, and the like.

“Aryl” and “arylene” refer to monovalent and divalent aromatic groups, respectively, including 5- and 6-membered monocyclic aromatic groups that contain 0 to 4 heteroatoms independently selected from nitrogen, oxygen, and sulfur. Examples of monocyclic aryl groups include, without limitation, phenyl, pyrrolyl, furanyl, thiopheneyl, thiazolyl, isothiazolyl, imidazolyl, triazolyl, tetrazolyl, pyrazolyl, oxazolyl, isooxazolyl, pyridinyl, pyrazinyl, pyridazinyl, pyrimidinyl, and the like. Aryl and arylene groups also include bicyclic groups, tricyclic groups, etc., including fused 5- and 6-membered rings described above. Examples of multicyclic aryl groups include, without limitation, naphthyl, biphenyl, anthracenyl, pyrenyl, carbazolyl, benzoxazolyl, benzodioxazolyl, benzothiazolyl, benzoimidazolyl, benzothiopheneyl, quinolinyl, isoquinolinyl, indolyl, benzofuranyl, purinyl, indolizinyl; and the like. They aryl and arylene groups may be attached to a parent group or to a substrate at any ring atom, unless such attachment would violate valence requirements. Likewise, aryl and arylene groups may include one or more non-hydrogen substituents unless such substitution would violate valence requirements. Useful substituents include, without limitation, alkyl, alkenyl, alkynyl, haloalkyl, haloalkenyl, haloalkynyl, cycloalkyl, cycloalkenyl, alkoxy, cycloalkoxy, alkanoyl, cycloalkanoyl, cycloalkenoyl, alkoxycarbonyl, cycloalkoxycarbonyl, and halo, as defined above, and hydroxy, mercapto, nitro, amino, and alkylamino.

“Heterocycle” and “heterocyclyl” refer to saturated, partially unsaturated, or unsaturated monocyclic or bicyclic rings having from 5 to 7 or from 7 to 11 ring members, respectively. These groups have ring members made up of carbon atoms and from 1 to 4 heteroatoms that are independently nitrogen, oxygen or sulfur, and may include any bicyclic group in which any of the above-defined monocyclic heterocycles are fused to a benzene ring. The nitrogen and sulfur heteroatoms may optionally be oxidized. The heterocyclic ring may be attached to a parent group or to a substrate at any heteroatom or carbon atom unless such attachment would violate valence requirements. Likewise, any of the carbon or nitrogen ring members may include a non-hydrogen substituent unless such substitution would violate valence requirements. Useful substituents include, without limitation, alkyl, alkenyl, alkynyl, haloalkyl, haloalkenyl, haloalkynyl, cycloalkyl, cycloalkenyl, alkoxy, cycloalkoxy, alkanoyl, cycloalkanoyl, cycloalkenoyl, alkoxycarbonyl, cycloalkoxycarbonyl, and halo, as defined above, and hydroxy, mercapto, nitro, amino, and alkylamino.

Examples of heterocycles include, without limitation, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

“Heteroaryl” and “heteroarylene” refer, respectively, to monovalent and divalent heterocycles or heterocyclyl groups, as defined above, which are aromatic. Heteroaryl and heteroarylene groups represent a subset of aryl and arylene groups, respectively.

“Arylalkyl” and “heteroarylalkyl” refer, respectively, to aryl-alkyl and heteroaryl-alkyl, where aryl, heteroaryl, and alkyl are defined above. Examples include, without limitation, benzyl, fluorenylmethyl, imidazol-2-yl-methyl, and the like.

“Arylalkanoyl,” “heteroarylalkanoyl,” “arylalkenoyl,” “heteroarylalkenoyl,” “arylalkynoyl,” and “heteroarylalkynoyl” refer, respectively, to aryl-alkanoyl, heteroaryl-alkanoyl, aryl-alkenoyl, heteroaryl-alkenoyl, aryl-alkynoyl, and heteroaryl-alkynoyl, where aryl, heteroaryl, alkanoyl, alkenoyl, and alkynoyl are defined above. Examples include, without limitation, benzoyl, benzylcarbonyl, fluorenoyl, fluorenylmethylcarbonyl, imidazol-2-oyl, imidazol-2-yl-methylcarbonyl, phenylethenecarbonyl, 1-phenylethenecarbonyl, 1-phenyl-propenecarbonyl, 2-phenyl-propenecarbonyl, 3-phenyl-propenecarbonyl, imidazol-2-yl-ethenecarbonyl, 1-(imidazol-2-yl)-ethenecarbonyl, 1-(imidazol-2-yl)-propenecarbonyl, 2-(imidazol-2-yl)-propenecarbonyl, 3-(imidazol-2-yl)-propenecarbonyl, phenylethynecarbonyl, phenylpropynecarbonyl, (imidazol-2-yl)-ethynecarbonyl, (imidazol-2-yl)-propynecarbonyl, and the like.

“Arylalkoxy” and “heteroarylalkoxy” refer, respectively, to aryl-alkoxy and heteroaryl-alkoxy, where aryl, heteroaryl, and alkoxy are defined above. Examples include, without limitation, benzyloxy, fluorenylmethyloxy, imidazol-2-yl-methyloxy, and the like.

“Aryloxy” and “heteroaryloxy” refer, respectively, to aryl-O— and heteroaryl-O—, where aryl and heteroaryl are defined above. Examples include, without limitation, phenoxy, imidazol-2-yloxy, and the like.

“Aryloxycarbonyl,” “heteroaryloxycarbonyl,” “arylalkoxycarbonyl,” and “heteroarylalkoxycarbonyl” refer, respectively, to aryloxy-C(O)—, heteroaryloxy-C(O)—, arylalkoxy-C(O)—, and heteroarylalkoxy-C(O)—, where aryloxy, heteroaryloxy, arylalkoxy, and heteroarylalkoxy are defined above. Examples include, without limitation, phenoxycarbonyl, imidazol-2-yloxycarbonyl, benzyloxycarbonyl, fluorenylmethyloxycarbonyl, imidazol-2-yl-methyloxycarbonyl, and the like.

“Leaving group” refers to any group that leaves a molecule during a fragmentation process, including substitution reactions, elimination reactions, and addition-elimination reactions. Leaving groups may be nucleofugal, in which the group leaves with a pair of electrons that formerly served as the bond between the leaving group and the molecule, or may be electrofugal, in which the group leaves without the pair of electrons. The ability of a nucleofugal leaving group to leave depends on its base strength, with the strongest bases being the poorest leaving groups. Common nucleofugal leaving groups include nitrogen (e.g., from diazonium salts); sulfonates, including alkylsulfonates (e.g., mesylate), fluoroalkylsulfonates (e.g., triflate, hexaflate, nonaflate, and tresylate); and arylsulfonates (e.g., tosylate, brosylate, closylate, and nosylate). Others include carbonates, halide ions, carboxylate anions, phenolate ions, and alkoxides. Some stronger bases, such as NH₂⁻ and OH⁻ can be made better leaving groups by treatment with an acid. Common electrofugal leaving groups include the proton, CO₂, and metals.

“Enantiomeric excess” or “ee” is a measure, for a given sample, of the excess of one enantiomer over a racemic sample of a chiral compound and is expressed as a percentage. Enantiomeric excess is defined as 100×(er−1)/(er+1), where “er” is the ratio of the more abundant enantiomer to the less abundant enantiomer.

“Diastereomeric excess” or “de” is a measure, for a given sample, of the excess of one diastereomer over a sample having equal amounts of diastereomers and is expressed as a percentage. Diastereomeric excess is defined as 100×(dr−1)/(dr+1), where “dr” is the ratio of a more abundant diastereomer to a less abundant diastereomer.

“Stereoselective,” “enantioselective,” “diastereoselective,” and variants thereof, refer to a given process (e.g., hydrogenation) that yields more of one stereoisomer, enantiomer, or diastereoisomer than of another, respectively.

“High level of stereoselectivity,” “high level of enantioselectivity,” “high level of diastereoselectivity,” and variants thereof, refer to a given process that yields products having an excess of one stereoisomer, enantiomer, or diastereoisomer, which comprises at least about 90% of the products. For a pair of enantiomers or diastereomers, a high level of enantioselectivity or diastereoselectivity would correspond to an ee or de of at least about 80%.

“Stereoisomerically enriched,” “enantiomerically enriched,” “diastereomerically enriched,” and variants thereof, refer, respectively, to a sample of a compound that has more of one stereoisomer, enantiomer or diastereomer than another. The degree of enrichment may be measured by % of total product, or for a pair of enantiomers or diastereomers, by ee or de.

“Substantially pure stereoisomer,” “substantially pure enantiomer,” “substantially pure diastereomer,” and variants thereof, refer, respectively, to a sample containing a stereoisomer, enantiomer, or diastereomer, which comprises at least about 95% of the sample. For pairs of enantiomers and diastereomers, a substantially pure enantiomer or diastereomer would correspond to samples having an ee or de of about 90% or greater.

A “pure stereoisomer,” “pure enantiomer,” “pure diastereomer,” and variants thereof, refer, respectively, to a sample containing a stereoisomer, enantiomer, or diastereomer, which comprises at least about 99.5% of the sample. For pairs of enantiomers and diastereomers, a pure enantiomer or pure diastereomer” would correspond to samples having an ee or de of about 99% or greater.

“Opposite enantiomer” refers to a molecule that is a non-superimposable mirror image of a reference molecule, which may be obtained by inverting all of the stereogenic centers of the reference molecule. For example, if the reference molecule has S absolute stereochemical configuration, then the opposite enantiomer has R absolute stereochemical configuration. Likewise, if the reference molecule has S,S absolute stereochemical configuration, then the opposite enantiomer has R,R stereochemical configuration, and so on.

“Stereoisomers” of a specified compound refer to the opposite enantiomer of the compound and to any diastereoisomers or geometric isomers (Z/E) of the compound. For example, if the specified compound has S,R,Z stereochemical configuration, its stereoisomers would include its opposite enantiomer having R,S,Z configuration, its diastereomers having S,S,Z configuration and R,R,Z configuration, and its geometric isomers having S,R,E configuration, R,S,E configuration, S,S,E configuration, and RRE configuration.

“Solvate” refers to a molecular complex comprising a disclosed or claimed compound and a stoichiometric or non-stoichiometric amount of one or more solvent molecules (e.g., EtOH).

“Hydrate” refers to a solvate comprising a disclosed or claimed compound and a stoichiometric or non-stoichiometric amount of water.

“Pharmaceutically acceptable complexes, salts, solvates, or hydrates” refers to complexes, acid or base addition salts, solvates or hydrates of claimed and disclosed compounds, which are within the scope of sound medical judgment, suitable for use in contact with the tissues of patients without undue toxicity, irritation, allergic response, and the like, commensurate with a reasonable benefit/risk ratio, and effective for their intended use.

“Pre-catalyst” or “catalyst precursor” refers to a compound or set of compounds that are converted into a catalyst prior to use.

“Treating” refers to reversing, alleviating, inhibiting the progress of, or preventing a disorder or condition to which such term applies, or to preventing one or more symptoms of such disorder or condition.

“Treatment” refers to the act of “treating,” as defined immediately above.

Table 1 lists abbreviations used throughout the specification.

TABLE 1
List of Abbreviations

| Abbreviation | Description |
|---|---|
| Ac | acetyl |
| ACN | acetonitrile |
| Ac₂O | acetic anhydride |
| aq | aqueous |
| (R,R)-BDPP | (2R,4R)-(+)-2,4-bis(diphenylphosphino)pentane |
| (R)-BICHEP | (R)-(−)-2,2′-bis(dicyclohexylphosphino)-6,6′-dimethyl-1,1′- |
| | biphenyl |
| (S,S)-BICP | (2S,2′S)-bis(diphenylphosphino)-(1S,1′S)-bicyclopentane |
| BIFUP | 2,2′-bis(diphenylphosphino)-4,4′,6,6′- |
| | tetrakis(trifluoromethyl)-1,1′-biphenyl |
| (R)-Tol-BINAP | (R)-(+)-2,2′-bis(di-p-tolylphosphino)-1,1′-binaphthyl |
| (S)-Tol-BINAP | (S)-(+)-2,2′-bis(di-p-tolylphosphino)-1,1′-binaphthyl |
| (R)-BINAP | (R)-2,2′-bis(diphenylphosphino)-1′1-binaphthyl |
| (S)-BINAP | (S)-2,2′-bis(diphenylphosphino)-1′1-binaphthyl |
| BIPHEP | 2,2′-bis(diphenylphosphino)-1,1′-biphenyl |
| (R)-MeO-BIPHEP | (R)-(6,6′-dimethoxybiphenyl-2,2′-diyl)- |
| | bis(diphenylphosphine) |
| (R)—Cl-MeO-BIPHEP | (R)-(+)-5,5′-dichloro-6,6′-dimethoxy-2,2′- |
| | bis(diphenylphosphino)-1,1′-biphenyl |
| (S)—Cl-MeO-BIPHEP | (S)-(+)-5,5′-dichloro-6,6′-dimethoxy-2,2′- |
| | bis(diphenylphosphino)-1,1′-biphenyl |
| BisP* | (S,S)-1,2-bis(t-butylmethylphosphino)ethane |
| (+)-tetraMeBITIANP | (S)-(+)-2,2′-bis(diphenylphosphino)-4,4′,6,6′-tetramethyl- |
| | 3,3′-bibenzo[b]thiophene |
| Bn | benzyl |
| BnBr, BnCl | benzylbromide, benzylchloride |
| Boc | t-butoxycarbonyl |
| BOP | benzotriazol-1-yloxy-tris-(dimethylamino)-phosphonium |
| | hexafluorophosphate |
| (R)—(S)-BPPFA | (−)-(R)—N,N-dimethyl-1-((S)-1′,2- |
| | bis(diphenylphosphino)ferrocenyl)ethylamine |
| (R,R)-Et-BPE | (+)-1,2-bis((2R,5i)-2,5-diethylphospholano)ethane |
| (R,R)-Me-BPE | (+)-1,2-bis((2R,5R)-2,5-dimethylphospholano)ethane |
| (S,S)-BPPM | (−)-(2S,4S)-2-diphenylphosphinomethyl-4- |
| | diphenylphosphino-1-t-butoxycarbonylpyrrolidine |
| Bs | brosyl or p-bromo-benzenesulfonyl |
| Bu | butyl |
| n-BuLi | n-butyl lithium |
| t-Bu | tertiary butyl |
| t-BuOK | potassium tertiary-butoxide |
| t-BuOLi | lithium tertiary-butoxide |
| (+)-CAMP | (R)-(+)-cyclohexyl(2-anisyl)methylphosphine; a |
| | monophosphine |
| CARBOPHOS | methyl-α-D-glucopyranoside-2,6-dibenzoate-3,4-di(bis(3,5- |
| | dimethylphenyl)phosphinite) |
| Cbz | benzyloxycarbonyl |
| CDI | N,N-carbonyldiimidazole |
| (S,S)-CHIRAPHOS | (2S,3S)-(−)-bis(diphenylphosphino)butane |
| CnTunaPHOS | 2,2′-bis-diphenylphosphanyl-biphenyl having an —O—(CH₂)ₙ—O— |
| | group linking the 6,6′ carbon atoms of the |
| | biphenyl (e.g., (R)-1,14-bis-diphenylphosphanyl-6,7,8,9- |
| | tetrahydro-5,10-dioxa-dibenzo[a,c]cyclodecene for n = 4). |
| (R)-CYCPHOS | (R)-1,2-bis(diphenylphosphino)-1-cyclohexylethane |
| DBAD | di-t-butyl azodicarboxylate |
| DBN | 1,5-diazabicyclo[4.3.0]non-5-ene |
| DBU | 1,8-diazabicyclo[5.4.0]undec-7-ene |
| DCC | dicycohexylcarbodiimide |
| de | diastereomeric excess |
| DEAD | diethyl azodicarboxylate |
| (R,R)-DEGUPHOS | N-benzyl-(3R,4R)-3,4-bis(diphenylphosphino)pyrrolidine |
| DIAD | diisopropyl azodicarboxylate |
| (R,R)-DIOP | (4R,5R)-(−)-O-isopropylidene-2,3-dihydroxy-1,4- |
| | bis(diphenylphosphino)butane |
| (R,R)-DIPAMP | (R,R)-(−)-1,2-bis[(O- |
| | methoxyphenyl)(phenyl)phosphino]ethane |
| DMAP | 4-(dimethylamino) pyridine |
| DMF | dimethylformamide |
| DMSO | dimethylsulfoxide |
| DMT-MM | 4-(4,6-dimethoxy-1,3,5-triazin-2-yl)-4-methylmorpholinium |
| | chloride |
| (R,R)-Et-DUPHOS | (−)-1,2-bis((2R,5R)-2,5-diethylphospholano)benzene |
| (S,S)-Et-DUPHOS | (−)-1,2-bis((2S,5S)-2,5-diethylphospholano)benzene |
| (R,R)-i-Pr-DUPHOS | (+)-1,2-bis((2R,5R)-2,5-di-i-propylphospholano)benzene |
| (R,R)-Me-DUPHOS | (−)-1,2-bis((2R,5R)-2,5-dimethylphospholano)benzene |
| (S,S)-Me-DUPHOS | (−)-1,2-bis((2S,5S)-2,5-dimethylphospholano)benzene |
| EDCI | 1-(3-dimethylaminopropyl)-3-ethylcarbodiimide |
| ee | enantiomeric excess |
| Et | ethyl |
| Et₃N | triethyl-amine |
| EtOAc | ethyl acetate |
| Et₂O | diethyl ether |
| EtOH | ethyl alcohol |
| FDPP | pentafluorophenyl diphenylphosphinate |
| (R,R)-Et-FerroTANE | 1,1′-bis((2R,4R)-2,4-diethylphosphotano)ferrocene |
| Fmoc | 9-fluoroenylmethoxycarbonyl |
| h, min, s | hour(s), minute(s), second(s) |
| HOAc | acetic acid |
| HOAt | 1-hydroxy-7-azabenzotriazole |
| HOBt | N-hydroxybenzotriazole |
| HODhbt | 3-hydroxy-3,4-dihydro-4-oxo-1,2,3-benzotriazine |
| (R)-(R)-JOSIPHOS | (R)-(−)-1-[(R)-2- |
| | (diphenylphosphino)ferrocenyl]ethyldicyclohexylphosphine |
| (S)—(S)-JOSIPHOS | (S)-(−)-1-[(S)-2- |
| | (diphenylphosphino)ferrocenyl]ethyldicyclohexylphosphine |
| (R)—(S)-JOSIPHOS | (R)-(−)-1-[(S)-2- |
| | (diphenylphosphino)ferrocenyl]ethyldicyclohexylphosphine |
| KHMDS | potassium hexamethyldisilazane |
| LDA | lithium diisopropylamide |
| LHMDS | lithium hexamethyldisilazane |
| LICA | lithium isopropylcyclohexylamide |
| LTMP | 2,2,6,6-tetramethylpiperidine |
| Me | methyl |
| MeCl₂ | methylene chloride |
| MEK | methylethylketone or butan-2-one |
| MeOH | methyl alcohol |
| (R,R)-t-butyl-miniPHOS | (R,R)-1,2-bis(di-t-butylmethylphosphino)methane |
| (S,S) MandyPhos | (S,S)-(−)-2,2′-bis[(R)—(N,N-dimethylamino) (phenyl)methyl]- |
| | 1,1′-bis(diphenylphosphino)ferrocene |
| (R)-MonoPhos | (R)-(−)-[4,N,N-dimethylamino]dinaphtho[2,1-d:1′,2′- |
| | f][1,3,2]dioxaphosphepin |
| (R)-MOP | (R)-(+)-2-(diphenylphosphino)-2′-methoxy-1,1′-binaphthyl |
| MPa | mega Pascals |
| mp | melting point |
| Ms | mesyl or methanesulfonyl |
| MTBE | methyl tertiary butyl ether |
| NMP | N-methylpyrrolidone |
| Ns | nosyl or nitrobenzene sulfonyl |
| (R,R)-NORPHOS | (2R,3R)-(−)-2,3-bis(diphenylphosphino)bicyclo[2.2.1]hept-5- |
| | ene |
| PdCl₂(dppf)₂ | dichloro[1,1′-bis(diphenylphosphino)ferrocene]palladium |
| | (II) dichloromethane adduct |
| (R,S,R,S)-Me- | (1R,2S,4R,5S)-2,5-dimethyl-7-phosphadicyclo[2.2.1]heptane |
| PENNPHOS | |
| Ph | phenyl |
| Ph₃P | triphenylphosphine |
| Ph₃As | triphenylarsine |
| (R)-PHANEPHOS | (R)-(−)-4,12-bis(diphenylphosphino)-[2.2]-paracyclophane |
| (S)-PHANEPHOS | (S)-(−)-4,12-bis(diphenylphosphino)-[2.2]-paracyclophane |
| (R)-PNNP | N,N′-bis[(R)-(+)-α-methylbenzyl]-N,N′- |
| | bis(diphenylphosphino)ethylene diamine |
| PPh₂-PhOx-Ph | (R)-(−)-2-[2-(diphenylphosphino)phenyl]-4-phenyl-2- |
| | oxazoline |
| Pr | propyl |
| i-Pr | isopropyl |
| (R)-PROPHOS | (R)-(+)-1,2-bis(diphenylphosphino)propane |
| PyBOP | benzotriazole-1-yl-oxy-tris-pyrrolidino-phosphonium |
| | hexafluorophosphate |
| (R)-QUINAP | (R)-(+)-1-(2-diphenylphosphino-1-naphthyl)isoquinoline |
| RT | room temperature (approximately 20° C. to 25° C.) |
| s/c | substrate-to-catalyst molar ratio |
| (R)-SpirOP | (1R,5R,6R)-spiro[4.4]nonane-1,6-diyl-diphenylphosphinous |
| | acid ester; a spirocyclic phosphinite ligand |
| (R,R,S,S) TangPhos | (R,R,S,S) 1,1′-di-t-butyl-[2,2′]biphospholanyl |
| TATU | O-(7-azabenzotriazol-1-yl)-1,1,3,3-tetramethyluronium |
| | tetrafluoroborate |
| (R)-eTCFP | (R)-2-{[(di-t-butyl-phosphanyl)-ethyl]-methyl-phosphanyl}- |
| | 2-methyl-propane |
| (S)-eTCFP | (S)-2-{[(di-t-butyl-phosphanyl)-ethyl]-methyl-phosphanyl}- |
| | 2-methyl-propane |
| (R)-mTCFP | (R)-2-{[(di-t-butyl-phosphanyl)-methyl]-methyl- |
| | phosphanyl}-2-methyl-propane |
| (S)-mTCFP | (S)-2-{[(di-t-butyl-phosphanyl)-methyl]-methyl- |
| | phosphanyl}-2-methyl-propane |
| TEA | triethanolamine |
| Tf | triflyl or trifluoromethylsulfonyl |
| TFA | trifluoroacetic acid |
| THF | tetrahydrofuran |
| TLC | thin-layer chromatography |
| TMS | trimethylsilyl |
| Tr | trityl or triphenylmethyl |
| Ts | tosyl or p-toluenesulfonyl |

Some of the schemes and examples below may omit details of common reactions, including oxidations, reductions, and so on, which are known to persons of ordinary skill in the art of organic chemistry. The details of such reactions can be found in a number of treatises, including Richard Larock, Comprehensive Organic Transformations (1999), and the multi-volume series edited by Michael B. Smith and others, Compendium of Organic Synthetic Methods (1974-2005). Starting materials and reagents may be obtained from commercial sources or may be prepared using literature methods.

In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and the like, see T. W. Greene and P. G. Wuts, Protecting Groups in Organic Chemistry (1999) and P. Kocienski, Protective Groups (2000), which are herein incorporated by reference in their entirety for all purposes.

Generally, the chemical transformations described throughout the specification may be carried out using substantially stoichiometric amounts of reactants, though certain reactions may benefit from using an excess of one or more of the reactants. Additionally, many of the reactions disclosed throughout the specification may be carried out at about RT, but particular reactions may require the use of higher temperatures (e.g., reflux conditions) or lower temperatures, depending on reaction kinetics, yields, and the like. Many of the chemical transformations may also employ one or more compatible solvents, which may influence the reaction rate and yield. Depending on the nature of the reactants, the one or more solvents may be polar protic solvents, polar aprotic solvents, non-polar solvents, or some combination. Any reference in the disclosure to a stoichiometric range, a temperature range, a pH range, etc., includes the indicated endpoints.

This disclosure concerns materials and methods for preparing optically active β-amino acids represented by Formula 1, above, including diastereomers thereof and pharmaceutically acceptable complexes, salts, solvates and hydrates thereof. The claimed and disclosed methods provide compounds of Formula 1 that are stereoisomerically enriched, and which in many cases, are pure or substantially pure stereoisomers.

The compounds of Formula 1 have at least two stereogenic centers and include substituents R¹, R², and R³. Substituents R¹ and R² are independently hydrogen atoms or C₁₋₃ alkyl optionally substituted with one to five fluorine atoms, provided that when R¹ is a hydrogen atom, R² is not a hydrogen atom. Substituent R³ is C₁₋₆ alkyl, C₃₋₆ cycloalkyl, C₃₋₆ cycloalkyl-C₁₋₆ alkyl, aryl, aryl-C₁₋₃ alkyl, or arylamino, wherein each alkyl of R³ is optionally substituted with one to five fluorine atoms, and each aryl of R³ is optionally substituted with from one to three substituents independently selected from chloro, fluoro, amino, nitro, cyano, C₁₋₃ alkylamino, C₁₋₃ alkyl optionally substituted with one to three fluorine atoms, and C₁₋₃ alkoxy optionally substituted with from one to three fluorine atoms.

Compounds of Formula 1 thus include those in which R¹ and R² are independently hydrogen or C₁₋₃ alkyl, provided that R¹ and R² are not both hydrogen, and those in which R³ is C₁₋₆ alkyl. Representative compounds of Formula 1 also include those in which R¹ is hydrogen, R² is methyl, and R³ is methyl, ethyl or n-propyl, i.e., (3S,5R)-3-amino-5-methyl-heptanoic acid, (3S,5R)-3-amino-5-methyl-octanoic acid, and (3S,5R)-3-amino-5-methyl-nonanoic acid.

Scheme I illustrates a method of preparing a desired stereoisomer of the compound of Formula 1. The stereoselective synthesis includes reacting an optically active β-dicarbonyl (Formula 6) with a source of ammonia to give an optically active enamine (Formula 4) that is optionally reacted with an acylating agent (Formula 5) to give an optically active enamide (Formula 2). The enamine (Formula 4) or the enamide (Formula 2) is reacted with hydrogen in the presence of a chiral catalyst to yield the compound of Formula 3, which is optionally hydrolyzed to the compound of Formula 1 by treatment with an acid or base. Substituents R¹, R², and R³ in Formula 2, 3, 4 and 6 are as defined in Formula 1; substituent R⁴ in Formula 2, 3, 4, and 6 is a hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl or a cation selected from a Group 1 metal ion, a Group 2 metal ion, a primary ammonium ion or a secondary ammonium ion; and substituents R⁵ in Formula 2 and Formula 5 and R¹⁹ in Formula 3 are independently hydrogen atom, carboxy, C₁₋₇ alkanoyl, C₂₋₇ alkenoyl, C₂₋₇ alkynoyl, C₃₋₇ cycloalkanoyl, C₃₋₇ cycloalkenoyl, halo-C₁₋₇ alkanoyl, halo-C₂₋₇ alkenoyl, halo-C₂₋₇ alkynoyl, C₁₋₆ alkoxycarbonyl, halo-C₁₋₆ alkoxycarbonyl, C₃₋₇ cycloalkoxycarbonyl, aryl-C₁₋₇ alkanoyl, aryl-C₂₋₇ alkenoyl, aryl-C₂₋₇ alkynoyl, aryloxycarbonyl, or aryl-C₁₋₆ alkoxycarbonyl, provided that R⁵ is not a hydrogen atom.

Generally, and unless stated otherwise, when a particular substituent identifier (R¹, R², R³, etc.) is defined for the first time in connection with a formula, the same substituent identifier, when used in a subsequent formula, will have the same definition as in the earlier formula. Thus, for example, if R³⁰ in a first formula is hydrogen atom, halogeno, or C₁₋₆ alkyl, then unless stated differently or otherwise clear from the context of the text, R³⁰ in a second formula is also hydrogen, halogeno, or C₁₋₆ alkyl.

The β-dicarbonyl (Formula 6) may be prepared using methods illustrated in Scheme IV and Scheme V, below, and is converted to the enamine (Formula 4) through treatment with an ammonia source. Representative β-dicarbonyl compounds (Formula 6) include various C₁₋₆ alkyl esters of (R)-5-methyl-3-oxo-heptanoic acid, (R)-5-methyl-3-oxo-octanoic acid, and (R)-5-methyl-3-oxo-nonanoic acid. Examples of β-dicarbonyls thus include (R)-5-methyl-3-oxo-heptanoic acid ethyl ester, (R)-5-methyl-3-oxo-octanoic, acid ethyl ester, and (R)-5-methyl-3-oxo-nonanoic acid ethyl ester. Useful sources of ammonia include ammonia and ammonium acetate, among others. See, e.g., P. G. Baraldi et al., Synthesis (11):902-903 (1983). The reaction is typically carried out with excess ammonium acetate (e.g., 1.2 eq. or greater) in a protic solvent, such as EtOH or HOAc, and at RT or above (up to reflux temperature).

As shown in Scheme I, the enamine (Formula 4) is optionally converted to the enamide (Formula 2) via contact with an acylating agent (Formula 5). Representative enamines include C₁₋₆ alkyl esters of the Z- and E-isomers of (R)-3-amino-5-methyl-hept-2-enoic acid, (R)-3-amino-5-methyl-oct-2-enoic acid, and (R)-3-amino-5-methyl-non-2-enoic acid. Examples of enamines thus include the Z- and E-isomers of (R)-3-amino-5-methyl-hept-2-enoic acid ethyl ester, (R)-3-amino-5-methyl-oct-2-enoic acid ethyl ester, and (R)-3-amino-5-methyl-non-2-enoic acid ethyl ester. Useful acylating agents include carboxylic acids, which have been activated either prior to contacting the enamine (Formula 4) or in-situ (i.e., in the presence of the enamine using an appropriate coupling agent). Representative activated carboxylic acids (Formula 5) include acid halides, anhydrides, mixed carbonates, and the like, in which X¹ is a leaving group, such as halogeno, aryloxy (e.g. phenoxy, 3,5-dimethoxyphenoxy, etc.) and heteroaryloxy (e.g., imidazolyloxy), or —OC(O)R¹⁵, in which R¹⁵ is C₁₋₆ alkyl, C₂₋₆ alkenyl, C₂₋₆ alkynyl, C₃₋₁₂ cycloalkyl, halo-C₁₋₆ alkyl, halo-C₂₋₆ alkenyl, halo-C₂₋₆ alkynyl, aryl, aryl-C₁₋₆ alkyl, heterocyclyl, heteroaryl, or heteroaryl-C₁₋₆ alkyl.

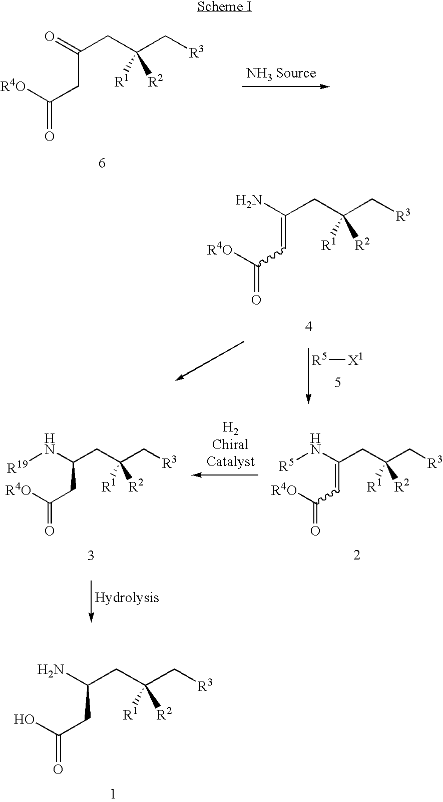

Other suitable acylating agents include carboxylic acids, which are activated in-situ using a coupling agent. Typically, the reaction is carried out in an aprotic solvent, such as ACN, DMF, DMSO, toluene, MeCl₂, NMP, THF, etc., and may also employ a catalyst. Coupling agents include, but are not limited to DCC, DMT-MM, FDPP, TATU, BOP, PyBOP, EDCI, diisopropyl carbodiimide, isopropenyl chloroformate, isobutyl chloroformate, N,N-bis-(2-oxo-3-oxazolidinyl)-phosphinic chloride, diphenylphosphoryl azide, diphenylphosphinic chloride, and diphenylphosphoryl cyanide. Useful catalysts for the coupling reaction include DMAP, HODhbt, HOBt, and HOAt.

The optically active enamine (Formula 4) or enamide (Formula 2) undergoes asymmetric hydrogenation in the presence of a chiral catalyst to give the compound of Formula 3. As depicted in Scheme I, useful enamide hydrogenation substrates (Formula 2) include individual Z- or E-isomers or a mixture of Z- and E-isomers, and include C₁₋₆ alkyl esters of the Z- and E-isomers of (R)-3-acetylamino-5-methyl-hept-2-enoic acid, (R)-3-acetylamino-5-methyl-oct-2-enoic acid, and (R)-3-acetylamino-5-methyl-non-2-enoic acid. Examples of useful enamides thus include the Z- and E-isomers of (R)-3-acetylamino-5-methyl-hept-2-enoic acid ethyl ester, (R)-3-acetylamino-5-methyl-oct-2-enoic acid ethyl ester, and (R)-3-acetylamino-5-methyl-non-2-enoic acid ethyl ester.

When substituent R⁴ in Formula 2 or 4 is a hydrogen atom, the method may optionally include converting the carboxylic acid to a Group 1, Group 2, or ammonium salt prior to asymmetric hydrogenation through contact with a suitable base, such as a primary amine (e.g., t-BuNH₂), a secondary amine (DIPEA), and the like. In some instances, the use of a salt of the enamide (Formula 2) or enamine (Formula 4) may increase conversion, improve stereoselectivity, or provide other advantages. Optionally, the method may employ an inorganic salt of the carboxylic acid obtained through contact with a suitable base such as NaOH, Na₂CO₂, LiOH, Ca(OH)₂, and the like.

Depending on which enantiomer of the chiral catalyst is used, the asymmetric hydrogenation generates an excess (de) of a diastereoisomer of Formula 3. Although the amount of the desired diastereoisomer produced will depend on, among other things, the choice of chiral catalyst, a de of the desired diastereoisomer of about 50% or greater is desirable; a de of about 70% or greater is more desirable; and a de of about 85% is still more desirable. Particularly useful asymmetric hydrogenations are those in which the de of the desired diastereoisomer is about 90% or greater. For the purposes of this disclosure, a desired diastereoisomer or enantiomer is considered to be substantially pure if it has a de or ee of 95% or greater.

As noted above, the asymmetric hydrogenation of the enamide (Formula 2) or enamine (Formula 4) employs a chiral catalyst having the requisite stereochemistry. Useful chiral catalysts include, without limitation, cyclic or acyclic, chiral phosphine ligands (e.g., monophosphines, bisphosphines, bisphospholanes, etc.) or phosphinite ligands bound to transition metals, such as ruthenium, rhodium, iridium or palladium. Ru-, Rh-, Ir- or Pd-phosphine, phosphinite or phosphino oxazoline complexes are optically active because they possess a chiral phosphorus atom or a chiral group connected to a phosphorus atom, or because in the case of BINAP and similar atropisomeric ligands, they possess axial chirality. Useful chiral ligands include, without limitation, BisP*; (R)-BINAPINE; (S)-Me-ferrocene-Ketalphos, (R,R)-DIOP; (R,R)-DIPAMP; (R)—(S)-BPPFA; (S,S)-BPPM; (+)-CAMP; (S,S)-CHIRAPHOS; (R)-PROPHOS; (R,R)-NORPHOS; (R)-BINAP; (R)-CYCPHOS; (R,R)-BDPP; (R,R)-DEGUPHOS; (R,R)-Me-DUPHOS; (R,R)-Et-DUPHOS; (R,R)-i-Pr-DUPHOS; (R,R)-Me-BPE; (R,R)-Et-BPE (R)-PNNP; (R)-BICHEP; (R,S,R,S)-Me-PENNPHOS; (S,S)-BICP; (R,R)-Et-FerroTANE; (R,R)-t-butyl-miniPHOS; (R)-Tol-BINAP; (R)-MOP; (R)-QUINAP; CARBOPHOS; (R)-(S)-JOSIPHOS; (R)-PHANEPHOS; BIPHEP; (R)—Cl-MeO-BIPHEP; (R)-MeO-BIPHEP; (R)-MonoPhos; BIFUP; (R)-SpirOP; (+)-TMBTP; (+)-tetraMeBITIANP; (R,R,S,S) TANGPhos; (R)-PPh₂-PhOx-Ph; (S,S) MandyPhos; (R)-eTCFP; (R)-mTCFP; and (R)-CnTunaPHOS, where n is an integer of 1 to 6.

Other useful chiral ligands include, without limitation, (R)-(−)-1-[(S)-2-(di(3,5-bistrifluoromethylphenyl)phosphino)ferrocenyl]ethyldicyclohexyl-phosphine; (R)-(−)-1-[(S)-2-(di(3,5-bis-trifluoromethylphenyl)phosphino)ferrocen-yl]ethyldi(3,5-dimethylphenyl)phosphine; (R)-(−)-1-[(S)-2-(di-t-butylphosphino)ferrocenyl]ethyldi(3,5-dimethylphenyl)phosphine; (R)-(−)-1-[(S)-2-(dicyclohexylphosphino)ferrocenyl]ethyldi-t-butylphosphine; (R)-(−)-1-[(S)-2-(dicyclohexylphosphino)ferrocenyl]ethyldicyclohexylphosphine; (R)-(−)-1-[(S)-2-(dicyclohexylphosphino)ferrocenyl]ethyldiphenylphosphine; (R)-(−)-1-[(S)-2-(di(3,5-dimethyl-4-methoxyphen-yl)phosphino)ferrocenyl]ethyldicyclohexylphosphine; (R)-(−)-1-[(5)-2-(diphenylphosphino)ferrocenyl]ethyldi-t-butylphosphine; (R)—N-[2-(N,N-dimethylamino)ethyl]-N-methyl-1-[(S)-1′,2-bis(diphenylphosphino)ferrocenyl]ethylamine; (R)-(+)-2-[2-(diphenylphosphino)phenyl]-4-(1-methylethyl)-4,5-dihydrooxazole; {1-[((R,R)-2-benzyl-phospholanyl)-phen-2-yl]-(R*,R*)-phospholan-2-yl}-phenyl-methane; and {1-[((R,R)-2-benzyl-phospholanyl)-ethyl]-(R*,R*)-phospholan-2-yl}-phenyl-methane.

Useful ligands may also include stereoisomers (enantiomers and diastereoisomers) of the chiral ligands described in the preceding paragraphs, which may be obtained by inverting all or some of the stereogenic centers of a given ligand or by inverting the stereogenic axis of an atropoisomeric ligand. Thus, for example, useful chiral ligands may also include (S)—Cl-MeO-BIPHEP; (S)-PHANEPHOS; (S,S)-Me-DUPHOS; (S,S)-Et-DUPHOS; (S)-BINAP; (S)-Tol-BINAP; (R)—(R)-JOSIPHOS; (S)—(S)-JOSIPHOS; (S)-eTCFP; (S)-mTCFP and so on.

Many of the chiral catalysts, catalyst precursors, or chiral ligands may be obtained from commercial sources or may be prepared using known methods. A catalyst precursor or pre-catalyst is a compound or set of compounds, which are converted into the chiral catalyst prior to use. Catalyst precursors typically comprise Ru, Rh, Ir or Pd complexed with the phosphine ligand and either a diene (e.g., norboradiene, COD, (2-methylallyl), etc.) or a halide (Cl or Br) or a diene and a halide, in the presence of a counterion, X⁻, such as OTf⁻, PF₆⁻, BF₄⁻, SbF₆⁻, ClO₄⁻, etc. Thus, for example, a catalyst precursor comprised of the complex, [(bisphosphine ligand)Rh(COD)]⁺X⁻ may be converted to a chiral catalyst by hydrogenating the diene (COD) in MeOH to yield [(bisphosphine ligand)Rh(MeOH)₂]⁺X⁻. MeOH is subsequently displaced by the enamide (Formula 2) or enamine (Formula 4), which undergoes enantioselective hydrogenation to the desired chiral compound (Formula 3). Examples of chiral catalysts or catalyst precursors include (+)-TMBTP-ruthenium(II) chloride acetone complex; (S)—Cl-MeO-BIPHEP-ruthenium(II) chloride Et₃N complex; (S)-BINAP-ruthenium(II) Br₂ complex; (S)-tol-BINAP-ruthenium(II) Br₂ complex; [((3R,4R)-3,4-bis(diphenylphosphino)-1-methylpyrrolidine)-rhodium-(1,5-cyclooctadiene)]-tetrafluoroborate complex; [((R,R,S,S)-TANGPhos)-rhodium(I)-bis(1,5-cyclooctadiene)]-trifluoromethane sulfonate complex; [(R)-BINAPINE-rhodium-(1,5-cyclooctaidene)]-tetrafluoroborate complex; [(S)-eTCFP-(1,5-cyclooctadiene)-rhodium(I)]-tetrafluoroborate complex; and [(S)-mTCFP-(1,5-cyclooctadiene)-rhodium(I)]-tetrafluoroborate complex.

For a given chiral catalyst and hydrogenation substrate (Formula 2 or 4), the molar ratio of the substrate and catalyst (s/c) may depend on, among other things, H₂ pressure, reaction temperature, and solvent (if any). Usually, the substrate-to-catalyst ratio exceeds about 100:1 or 200:1, and substrate-to-catalyst ratios of about 1000:1 or 2000:1 are common. Although the chiral catalyst may be recycled, higher substrate-to-catalyst ratios are more useful. For example, substrate-to-catalyst ratios of about 1000:1, 10,000:1, and 20,000:1, or greater, would be useful. The asymmetric hydrogenation is typically carried out at about RT or above, and under about 10 kPa (0.1 atm) or more of H₂. The temperature of the reaction mixture may range from about 20° C. to about 80° C., and the H₂ pressure may range from about 10 kPa to about 5000 kPa or higher, but more typically, ranges from about 10 kPa to about 100 kPa. The combination of temperature, H₂ pressure, and substrate-to-catalyst ratio is generally selected to provide substantially complete conversion (i.e., about 95 wt %) of the substrate (Formula 2 or 4) within about 24 h. With many of the chiral catalysts, decreasing the H₂ pressure increases the enantioselectivity.

A variety of solvents may be used in the asymmetric hydrogenation, including protic solvents, such as water, MeOH, EtOH, and i-PrOH. Other useful solvents include aprotic polar solvents, such as THF, ethyl acetate, and acetone. The stereoselective hydrogenation may employ a single solvent or may employ a mixture of solvents, such as THF and MeOH, THF and water, EtOH and water, MeOH and water, and the like.

In some cases it may be advantageous to employ more than one chiral catalyst to carryout the asymmetric hydrogenation of the substrate (Formula 2 or 4). For example, the method may provide for reacting the enamide or enamine successively with first and second chiral catalysts to exploit the comparatively greater stereoselectivity, but lower reaction rate of the first (or second) chiral catalyst. Thus, for example, the method provides for reacting the substrate with hydrogen in the presence of a chiral catalyst comprised of (R)-BINAPINE or its opposite enantiomer, followed by reaction in the presence of a chiral catalyst comprised of (R)-mTCFP or its opposite enantiomer.

As shown in Scheme I, the method optionally provides for conversion of the hydrogenation product (Formula 3) into the optically active β-amino acid (Formula 1). For example, when R⁴ is C₁₋₆ alkyl and R¹⁹ is non-hydrogen, the ester and amide moieties may be hydrolyzed by treatment with an acid or a base or by treatment with a base (or acid) followed by treatment with an acid (or base). For example, treating the compound of Formula 3 with HCl, H₂SO₄, and the like, with excess H₂O generates the β-amino acid (Formula 1) or an acid addition salt. Treating the compound of Formula 3 with an aqueous inorganic base, such as LiOH, KOH, NaOH, CsOH, Na₂CO₃, K₂CO₃, CS₂CO₃, and the like, in an optional polar solvent (e.g., THF, MeOH, EtOH, acetone, ACN, etc.) gives a base addition salt of a β-amido acid, which may be treated with an acid to generate the β-amino acid (Formula 1) or an acid addition salt. Likewise, when R¹⁹ in Formula 3 is hydrogen, the ester moiety may be hydrolyzed by treatment with an acid or base to give the β-amino acid (Formula 1) or an acid or base addition salt. The ester and amide hydrolysis may be carried out at RT or at temperatures up to reflux temperature, and if desired, treatment of the acid or base addition salts with a suitable base (e.g., NaOH) or acid (e.g., HCl) gives the free amino acid (zwitterion).

Useful compounds represented by Formula 3 include β-amino and β-amido C₁₋₆ alkyl esters in which R¹ and R¹ are independently hydrogen or C₁₋₃ alkyl, provided that R¹ and R² are not both hydrogen, and those in which R³ is C₁₋₄ alkyl. Useful compounds of Formula 3 also include those in which R¹ is hydrogen, R² is methyl, and R³ is methyl, ethyl or n-propyl, i.e., C₁₋₆ alkyl esters of (3S,5R)-3-amino-5-methyl-heptanoic acid, (3S,5R)-3-amino-5-methyl-octanoic acid, (3S,5R)-3-amino-5-methyl-nonanoic acid, (3S,5R)-3-acetylamino-5-methyl-heptanoic acid, (3S,5R)-3-acetylamino-5-methyl-octanoic acid, and (3S,5R)-3-acetylamino-5-methyl-nonanoic acid. Examples of useful β-amino C₁₋₆ alkyl esters thus include (3S,5R)-3-amino-5-methyl-heptanoic acid ethyl ester, (3S,5R)-3-amino-5-methyl-octanoic acid ethyl ester, and (3S,5R)-3-amino-5-methyl-nonanoic acid ethyl ester. Likewise, useful β-amido C₁₋₆ alkyl esters include (3S,5R)-3-acetylamino-5-methyl-heptanoic acid ethyl ester, (3S,5R)-3-acetylamino-5-methyl-octanoic acid ethyl ester, and (3S,5R)-3-acetylamino-5-methyl-nonanoic acid ethyl ester.

Compounds of Formula 3 also include β-amido acids in which R¹ and R² are independently hydrogen or C₁₋₃ alkyl, provided that R¹ and R² are not both hydrogen, and those in which R³ is C₁₋₆ alkyl. Useful β-amido acids of Formula 3 also include those in which R¹ is hydrogen, R² is methyl, and R³ is methyl, ethyl or n-propyl, i.e., (3S,5R)-3-acetylamino-5-methyl-heptanoic acid, (3S,5R)-3-acetylamino-5-methyl-octanoic acid, and (3S,5R)-3-acetylamino-5-methyl-nonanoic acid.

The compound of Formula 1, or its diastereoisomer, may be further enriched through, e.g., fractional recrystallization or chromatography or by recrystallization in a suitable solvent. In addition, compounds of Formula 1 or 3 may be enriched through treatment with an enzyme such as a lipase or amidase.

Scheme II illustrates another method for preparing the desired stereoisomer of the compound of Formula 1. The stereoselective synthesis includes reacting an optically active β-dicarbonyl (Formula 6) with hydrogen in the presence of a chiral catalyst to yield an optically active β-hydroxy carboxylic acid derivative (Formula 12) that is subsequently hydrolyzed to give the corresponding β-hydroxy acid (Formula 10). The activated acid is reacted with an amine (Formula 11) to give an optically active amide (Formula 9), which is cyclized under Mitsunobu conditions (e.g., Ph₃P, DEAD, dry THF) to give a chiral lactam (Formula 8) with inversion of the stereocenter. Besides DEAD, other useful azodicarboxylates include DBAD, DIAD, and 1,1′-(azodicarbonyl)dipiperidine. In addition to Mitsunobu conditions, the alcohol (Formula 9) may be activated by conversion to a sulfonate ester (e.g., reaction with MsCl and pyridine), which is subsequently cyclized by treatment with a base (e.g., a carbonate). Treatment of the lactam (Formula 8) with an acid or base gives a secondary amine (Formula 7), which is subsequently reduced via, e.g., catalytic hydrogenolysis to give the compound of Formula 1. Substituents R¹, R², and R³ in Formula 7 to 10 and Formula 12 are as defined in Formula 1; substituent R⁴ in Formula 12 is as defined in Formula 6; and substituent R⁶ in Formula 7 to 9 and Formula 11 is aryl-C₁₋₃ alkyl (e.g., benzyl, 3,5-dimethoxybenzyl, etc.), C₁₋₆ alkyl (e.g., methyl) or C₂₋₆ alkenyl (e.g., allyl).

The methodology shown in Scheme II may employ many of the same reagents and conditions described in Scheme I. For example, useful reagents (substrates, chiral catalysts, solvents, etc.) and conditions (temperature, pressure, etc.) for the stereoselective hydrogenation of the β-dicarbonyl (Formula 6) to give the β-hydroxy carboxylic acid derivative (Formula 12) include the reagents and conditions described in Scheme I for the asymmetric hydrogenation of the enamide (Formula 2). However, because the formation of the lactam (Formula 8) inverts the β-carbon stereocenter, the chiral catalyst should promote the formation of a hydroxy-substituted stereocenter (Formula 12) having the opposite stereochemical configuration as that of the β-carbon of the final product (Formula 1).

Similarly, reagents and conditions for coupling the β-hydroxy carboxylic acid (Formula 10) and the primary amine (Formula 11) to give the chiral amide (Formula 9) include reagents and conditions described in Scheme I for acylation of the enamine (Formula 4). For example, the β-hydroxy carboxylic acid (Formula 10) may be activated in-situ with a coupling agent (e.g., DMT-MM) and reacted with a primary amine (e.g., BnONH₂ or BnONH₃⁺Cl) to give the chiral amide (Formula 9 in which R⁶ is Bn). Useful β-hydroxy carboxylic acids (Formula 10) include (3R,5R)-3-hydroxy-5-methyl-heptanoic acid, (3R,5R)-3-hydroxy-5-methyl-octanoic acid, and (3R,5R)-3-hydroxy-5-methyl-nonanoic acid. Representative β-hydroxy amides (Formula 9) include aryl-C₁₋₃ alkyl amides derived from the aforementioned carboxylic acids, including (3R,5R)-3-hydroxy-5-methyl-heptanoic acid benzyloxy-amide, (3R,5R)-3-hydroxy-5-methyl-octanoic acid benzyloxy-amide, and (3R,5R)-3-hydroxy-5-methyl-nonanoic acid benzyloxy-amide.

Likewise, reagents and conditions for hydrolyzing the β-hydroxy carboxylic acid derivative (Formula 12) or the lactam (Formula 8) include reagents and conditions described in Scheme I for hydrolysis of the amino acid ester (Formula 3). Useful, β-hydroxy carboxylic acid derivatives (Formula 12) include C₁₋₆ alkyl esters of (3R,5R)-3-hydroxy-5-methyl-heptanoic acid, (3R,5R)-3-hydroxy-5-methyl-octanoic acid, and (3R,5R)-3-hydroxy-5-methyl-nonanoic acid. Examples of useful β-hydroxy C₁₋₆ alkyl esters include (3R,5R)-3-hydroxy-5-methyl-heptanoic acid ethyl ester, (3R,5R)-3-hydroxy-5-methyl-octanoic acid ethyl ester, and (3R,5R)-3-hydroxy-5-methyl-nonanoic acid ethyl ester. Representative lactams (Formula 8) include (2R,4S)-1-(aryl-C₁₋₃ alkyloxy)-4-(2-methyl-butyl)-azetidin-2-one, (2R,4S)-1-(aryl-C₁₋₃ alkyloxy)-4-(2-methyl-butyl)-azetidin-2-one, and (2R,4S)-1-(aryl-C₁₋₃ alkyloxy)-4-(2-methyl-butyl)-azetidin-2-ones. These include, for example, (2R,4S)-1-benzyloxy-4-(2-methyl-butyl)-azetidin-2-one, (2R,4S)-1-benzyloxy-4-(2-methyl-pentyl)-azetidin-2-one, and (2R,4S)-1-benzyloxy-4-(2-methyl-hexyl)-azetidin-2-one.

Hydrogenolysis is carried out in the presence of a catalyst and one or more polar solvents, including without limitation, alcohols, ethers, esters and acids, such as MeOH, EtOH, IPA, THF, EtOAc, and HOAc. The reaction may be carried out at temperatures ranging from about 5° C. to about 100° C., though reactions at RT are common. Generally, the substrate-to-catalyst ratio may range from about 1:1 to about 1000:1, based on weight, and H₂ pressure may range from about atmospheric pressure, 0 psig, to about 1500 psig. More typically, the substrate-to-catalyst ratios range from about 4:1 to about 20:1, and H₂ pressures range from about 25 psig to about 150 psig.

Useful substrates (Formula 7) include those in which R¹ and R² are independently hydrogen or C₁₋₃ alkyl, provided that R¹ and R² are not both hydrogen, and those in which R³ is C₁₋₆ alkyl. Representative compounds of Formula 7 include those in which R¹ is hydrogen, R² is methyl, R³ is methyl, ethyl or n-propyl, and R⁶ is benzyl, i.e., (3S,5R)-3-benzyloxyamino-5-methyl-heptanoic acid, (3S,5R)-3-benzyloxyamino-5-methyl-octanoic acid, and (3S,5R)-3-benzyloxyamino-5-methyl-nonanoic acid.

Useful catalysts include, without limitation, heterogeneous catalysts containing from about 0.1% to about 20%, and more typically, from about 1% to about 5%, by weight, of transition metals such as Ni, Pd, Pt, Rh, Re, Ru, and Ir, including oxides and combinations thereof, which are typically supported on various materials, including Al₂O₃, C, CaCO₃, SrCO₃, BaSO₄, MgO, SiO₂, TiO₂, ZrO₂, and the like. Many of these metals, including Pd, may be doped with an amine, sulfide, or a second metal, such as Pb, Cu, or Zn. Useful catalysts thus include palladium catalysts such as Pd/C, Pd/SrCO₃, Pd/Al₂O₃, Pd/MgO, Pd/CaCO₃, Pd/BaSO₄, PdO, Pd black, PdCl₂, and the like, containing from about 1% to about 5% Pd, based on weight. Other useful catalysts include Raney nickel, Rh/C, Ru/C, Re/C, PtO₂, Rh/C, RuO₂, and the like. For a discussion of hydrogenolysis catalysts, see U.S. Pat. No. 6,624,112 to Hasegawa et al., which is herein incorporated by reference.

Scheme III illustrates an additional method for preparing the desired stereoisomer of the compound of Formula 1. The stereoselective synthesis includes reducing an optically active β-dicarbonyl (Formula 6) by, e.g., reacting it with hydrogen in the presence of a metal catalyst, to give an optically active β-hydroxy carboxylic acid derivative (Formula 17). Activating the β-hydroxy moiety via, e.g., reaction with a compound of Formula 18, gives an intermediate (Formula 16), which undergoes elimination via treatment with a base. Reacting the resulting unsaturated acid derivative (Formula 15) with an anion of a chiral amine (Formula 14) gives, after protonation, an optically active secondary or tertiary amine (Formula 13), which is subsequently deprotected via catalytic hydrogenolysis to give the compound of Formula 37. As in Scheme I, the compound of Formula 37 is optionally hydrolyzed to the compound of Formula 1 by treatment with an acid or base. Substituents R¹, R², and R³ and substituent R⁴ in Formula 13, 15 to 17, and 37 are as defined in Formula 1 and Formula 6, respectively; substituent R⁷ in Formula 13 and 14 is C₁₋₆ alkyl, C₂₋₆ alkenyl, or aryl-C₁₋₃ alkyl; substituent R⁸ in Formula 16 is a leaving group (e.g., R⁹O—); substituent R⁹ in Formula 18 is tosyl, mesyl, brosyl, closyl (p-chloro-benzenesulfonyl), nosyl, or triflyl; and substituent X² in Formula 18 is halogeno or R⁹O—.

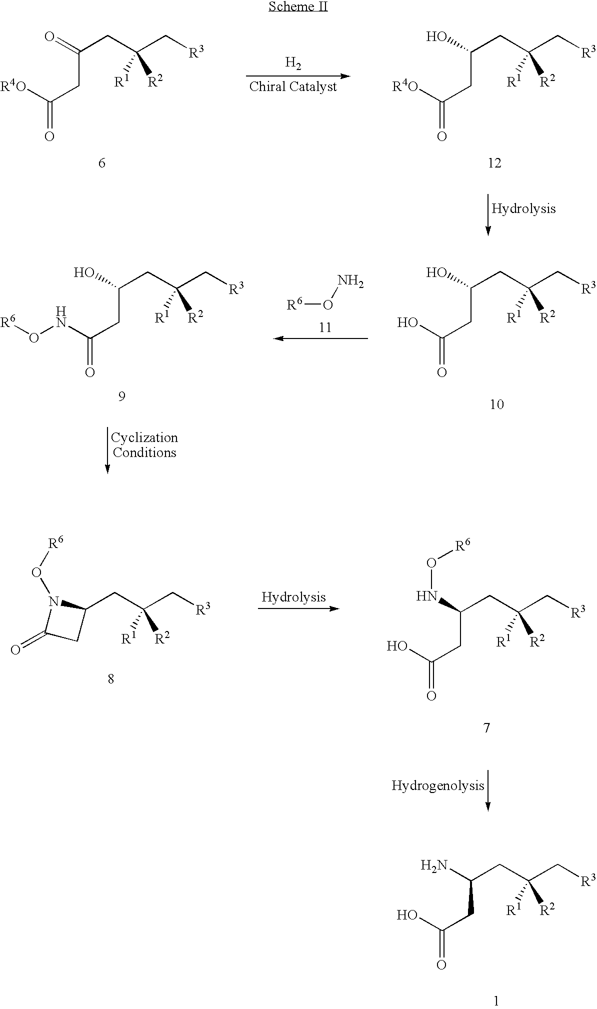

The methodology shown in Scheme III may employ many of the same reagents and conditions described in Scheme II. For example, useful reagents (catalysts, solvents, etc.) and conditions (temperature, pressure, etc.) for the catalytic reduction of the β carbonyl and substituted amino moieties of the compounds of Formula 6 and Formula 13, respectively, include the reagents and conditions described in Scheme II for the catalytic hydrogenolysis of the secondary amine (Formula 7). Representative optically active secondary or tertiary amines (Formula 13) include β-amino C₁₋₆ alkyl esters of (1S,3S,5R)-3-[benzyl-(1-phenyl-ethyl)-amino]-5-methyl-heptanoic acid, (1S,3S,5R)-3-[benzyl-(1-phenyl-ethyl)-amino]-5-methyl-octanoic acid, and (1S,3S,5R)-3-[benzyl-(1-phenyl-ethyl)-amino]-5-methyl-nonanoic acid. Examples of useful β-amino C₁₋₆ alkyl esters thus include (1S,3S,5R)-3-[benzyl-(1-phenyl-ethyl)-amino]-5-methyl-heptanoic acid ethyl ester, (1S,3S,5R)-3-[benzyl-(1-phenyl-ethyl)-amino]-5-methyl-octanoic acid ethyl ester, and (1S,3S,5R)-3-[benzyl-(1-phenyl-ethyl)-amino]-5-methyl-nonanoic acid ethyl ester.

As shown in Scheme III, the β-hydroxy moiety of the compound of Formula 17 is activated via reaction with the compound of Formula 18. Useful β-hydroxy carboxylic acid derivatives (Formula 17) include C₁₋₆ alkyl esters of (5R)-3-hydroxy-5-methyl-heptanoic acid, (5R)-3-hydroxy-5-methyl-octanoic acid, and (5R)-3-hydroxy-5-methyl-nonanoic acid. Representative β-hydroxy C₁₋₆ alkyl esters thus include (5R)-3-hydroxy-5-methyl-heptanoic acid ethyl ester, (5R)-3-hydroxy-5-methyl-octanoic acid ethyl ester, and (5R)-3-hydroxy-5-methyl-nonanoic acid ethyl ester.

Useful compounds of Formula 18 include sulfonylating agents, such as TsCl, MsCl, BsCl, NsCl, TfCl, and the like, and their corresponding anhydrides (e.g., p-toluenesulfonic acid anhydride). Thus, for example, compounds of Formula 17 may be reacted with TsCl in the presence of pyridine and an aprotic solvent, such as ethyl acetate, MeCl₂, ACN, THF, and the like, to give C₁₋₆ alkyl esters of (5R)-5-methyl-3-(toluene-4-sulfonyloxy)-heptanoic acid, (5R)-5-methyl-3-(toluene-4-sulfonyloxy)-octanoic acid, and (5R)-5-methyl-3-(toluene-4-sulfonyloxy)-nonanoic acid. Likewise, compounds of Formula 17 may be reacted with MsCl in the presence of a an aprotic solvent and a strong or hindered base, such as Et₃N, to give C₁₋₆ alkyl esters of (5R)-3-methanesulfonyloxy-5-methyl-heptanoic acid, (5R)-3-methanesulfonyloxy-5-methyl-octanoic acid, and (5R)-3-methanesulfonyloxy-5-methyl-nonanoic acid.

Upon activation of the β-hydroxy moiety, the resulting intermediate (Formula 16) is reacted with a base to give an unsaturated carboxylic acid derivative (Formula 15). The reaction is typically carried out at RT or above and in the presence of an aprotic solvent, such as ethyl acetate, THF, MeCl₂, ACN, and the like. Useful bases include strong or hindered bases (i.e., non-nucleophilic bases) such as Et₃N, t-BuOK, DBN, DBU, and the like.

As indicated above, conjugate addition of a chiral amine (Formula 14) to an unsaturated-carboxylic acid derivative (Formula 15) gives an optically active secondary or tertiary amine (Formula 13). The stereochemistry of the chiral amine (Formula 14) determines the stereochemical configuration of the amino-substituted stereocenter (Formula 13). Useful substrates for the conjugate addition include Z- or E-isomers or a mixture of Z- and E-isomers of the unsaturated carboxylic acid derivative (Formula 15) and include C₁₋₆ alkyl esters of the Z- and E-isomers of (R)-5-methyl-hept-2-enoic acid, (R)-5-methyl-oct-2-enoic acid, and (R)-5-methyl-non-2-enoic acid. Examples include the Z- and E-isomers of (R)-5-methyl-hept-2-enoic acid ethyl ester, (R)-5-methyl-oct-2-enoic acid ethyl ester, and (R)-5-methyl-non-2-enoic acid ethyl ester. Useful chiral amines (Formula 14) include (R)-(+)-N-benzyl-α-methylbenzylamine, (S)-(−)-N-benzyl-α-methylbenzylamine, and the like. See S. G. Davies and O. Ichihara, Tetrahedron: Asymmetry 2(3):183-186 (1991); and J. Chem. Soc., Perkins Trans. 1 2931-2938 (2001).

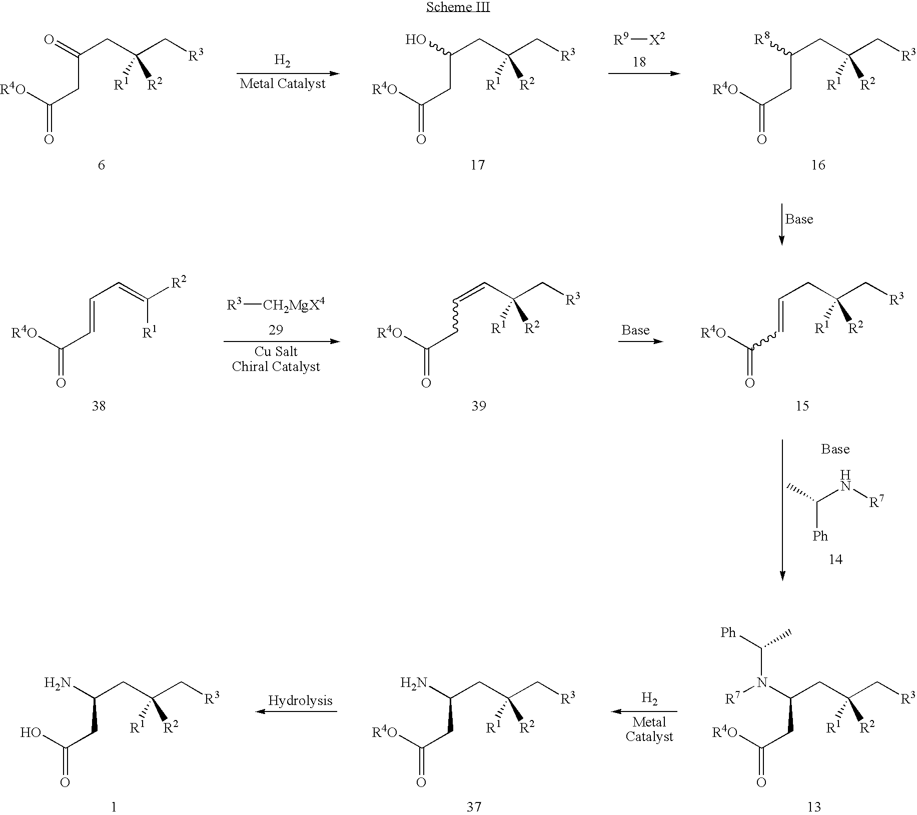

To carryout the conjugate addition, the chiral amine (Formula 14) is typically treated with a strong base, such as n-BuLi and the like, in an ethereal solvent, such as Et₂O, THF, etc., and at a temperature of about −78° C. to RT. The resulting deprotonated amine is subsequently reacted with the unsaturated carboxylic acid derivative (Formula 15) to give the optically active secondary or tertiary amine (Formula 13) having the desired stereochemical configuration.

Scheme III shows an alternative method for preparing the compound of Formula 15. The method includes reacting a sorbate ester (Formula 38) or amide with a Grignard reagent (Formula 29) and a catalytic amount of another metal (e.g., copper salt) and an optional chiral catalyst. The resulting enantiomerically enriched compound (Formula 39) is subsequently isomerized by treatment with a base (e.g., triethylamine) in a polar solvent (e.g., THE) to give the compound of Formula 15. Alternatively, the compound of Formula 39 may be isomerized by treatment with a metal catalyst, including Ru, Rh or Pd salts complexed with a counterion, such as a halogen anion, COD, and the like. Compounds of Formula 15 and 39 may also be enantiomerically enriched by treatment with a lipase under standard conditions. The optional chiral catalysts may include those described above in connection with the asymmetric hydrogenation of the enamide (Formula 2) and enamine (Formula 4) in Scheme I.

Substituents R¹, R², and R³ and substituent R⁴ in Formula 29, 38 and 39 are as defined in Formula 1 and Formula 6, respectively, and substituent X⁴ in Formula 29 is halogeno. Useful compounds of Formula 39 thus include, without limitation, C₁₋₆ alkyl esters of the Z and E-isomers of (R)-5-methyl-hept-3-enoic acid, (R)-5-methyl-oct-3-enoic acid, and (R)-5-methyl-non-3-enoic acid. Examples include the Z- and E-isomers of (R)-5-methyl-hept-3-enoic acid ethyl ester, (R)-5-methyl-oct-3-enoic acid ethyl ester, and (R)-5-methyl-non-3-enoic acid ethyl ester.

In addition to the methodology shown in Scheme III, the compound of Formula 37 may be prepared from the compound of Formula 15 by catalytic asymmetric conjugate addition of an amine. See, e.g., Hamashima et al., Organic Letters 6:1861-1864 (2004), the complete disclosure of which is herein incorporated by reference.

Scheme IV illustrates a method for preparing β-dicarbonyls (Formula 6) used in Scheme I, II, and III. The method includes activating a chiral alcohol (Formula 25) via, e.g., reaction with a sulfonylating agent (Formula 26), to give an intermediate (Formula 24), which is subsequently treated with a source of cyanide ion to yield an optically active nitrite (Formula 23). Hydrolyzing the nitrite (Formula 23) through contact with an acid gives a chiral carboxylic acid (Formula 22 or salt), which is subsequently activated through, e.g., reaction with a coupling agent such as CDI. The activated carboxylic acid derivative (Formula 20) is reacted with a malonic acid salt or ester (Formula 21) in the presence of a base to give an α-substituted malonic acid intermediate (Formula 19), which is decarboxylated by treatment with an acid to provide the desired β-dicarbonyl (Formula 6). Useful sulfonylating agents include those described in connection with Formula 18; useful sources of cyanide ion include, without limitation, sodium cyanide, potassium cyanide, zinc cyanide, hydrogen cyanide, acetone cyanohydrin, and the like, either alone or in combination.

Instead of the chiral alcohol (Formula 25), the method may employ an activated prochiral enoate (Formula 30), which is reacted with a deprotonated chiral oxazolidinone to give an N-acylated oxazolidinone (Formula 28). The deprotonated oxazolidinone may be prepared from a chiral oxazolidinone (Formula 31) by separate treatment with a strong base (e.g., n-BuLi) or by in-situ treatment with a hindered base (e.g., Et₃N). The N-acylated oxazolidinone (Formula 28) is subsequently reacted with a Grignard reagent (Formula 29) in the presence of a copper salt (e.g., CuBrDMS) to give a conjugate addition product (Formula 27). As indicated in Scheme IV, the conjugate addition product (Formula 27 or Formula 20 in which R¹⁰ is a chiral oxazolidin-2-one-3-yl) may be reacted with the malonic acid derivative (Formula 21) to give the α-substituted malonic acid intermediate (Formula 19). Alternatively, the chiral side chain may be cleaved following asymmetric synthesis via, e.g., acid or base hydrolysis, using for instance, an alkali metal hydroxide or peroxide such as LiOOH in aq. THF followed by reduction, to give the carboxylic acid of Formula 22 or a salt thereof and to regenerate the chiral auxiliary (Formula 31). For additional methods of cleaving the chiral side, see U.S. Pat. No. 5,801,249 to Davies et al. and references cited therein.

In Scheme IV, substituents R¹, R², and R³ in Formula 19, 20, 22 to 25, and 27, 28 and 30 are as defined above in Formula 1; R⁴ in Formula 19 and 21 is as defined in Formula 2; R¹⁰, R¹¹, and R¹⁷ in Formula 20, 24, and 30 are leaving groups, which may be the same or different; R¹² in Formula 26 is a tosyl, mesyl, brosyl, closyl, nosyl, or triflyl; X³ in Formula 26 is halogeno; and R¹³, R¹⁴, R¹⁵, and R¹⁶ in Formula 27, 28, and 31 are independently hydrogen atom, C₁₋₆ alkyl, C₃₋₆ cycloalkyl, C₃₋₆ cycloalkyl-C₁₋₆ alkyl, aryl, or aryl-C₁₋₃ alkyl, provided that R¹⁵ and R¹⁶ are not both hydrogen atoms.

For the methodology shown in Scheme IV, representative chiral alcohols (Formula 25) and corresponding activated forms (Formula 24) include, without limitation, (R)-2-methyl-butanol, (R)-2-methyl-pentanol, (R)-2-methyl-hexanol, (R)-2-methyl-1-(toluene-4-sulfonyloxy)-butane, (R)-2-methyl-1-(toluene-4-sulfonyloxy)-pentane, (R)-2-methyl-1-(toluene-4-sulfonyloxy)-hexane, (R)-1-methanesulfonyloxy-2-methyl-butane, (R)-1-methanesulfonyloxy-2-methyl-pentane, and (R)-1-methanesulfonyloxy-2-methyl-hexane. Representative nitriles (Formula 23), chiral carboxylic acids (Formula 22) and corresponding activated forms (Formula 20) include, without limitation, (R)-3-methyl-pentanenitrile, (R)-3-methyl-hexanenitrile, (R)-3-methyl-heptanenitrile, (R)-1-imidazol-1-yl-3-methyl-pentan-1-one, (R)-1-imidazol-1-yl-3-methyl-hexan-1-one, and (R)-1-imidazol-1-yl-3-methyl-heptan-1-one.

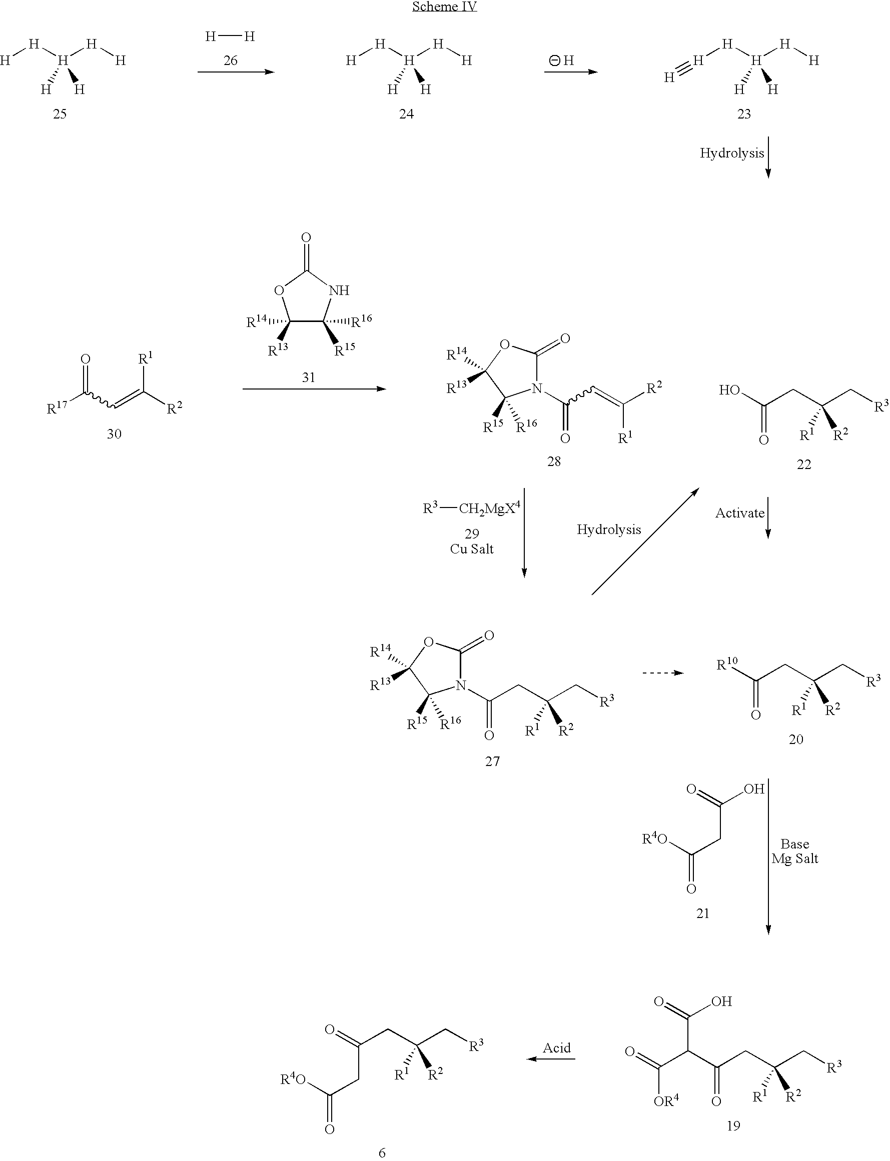

In addition, representative activated prochiral enoates (Formula 30) include, without limitation, acid halides of the Z- and E-isomers of but-2-enoic acid, such as but-2-enoyl chloride. Representative chiral oxazolidinones (Formula 31) include, without limitation, (R) -isopropyl-oxazolidin-2-one, (R)-4-phenyl-oxazolidin-2-one, (R) -4-benzyl-oxazolidin-2-one, and (4R,5S)-4-methyl-5-phenyl-oxazolidin-2-one. Thus, representative N-acylated oxazolidinones (Formula 28) include, without limitation, the Z- and E-isomers of (R)-3-(but-2-enoyl)-4-isopropyl-oxazolidin-2-one, (R)-3-(but-2-enoyl)-4-phenyl-oxazolidin-2-one, (R)-4-benzyl-3-(but-2-enoyl)-oxazolidin-2-one, and (4R,5S)-3-(but-2-enoyl)-4-methyl-5-phenyl-oxazolidin-2-one. Likewise, representative Michael adducts (Formula 27 or Formula 20) include, without limitation, (R,R)-4-isopropyl-3-(3-methyl-pentanoyl)-oxazolidin-2-one, (R,R)-4-isopropyl-3-(3-methyl-hexanoyl)-oxazolidin-2-one, (R,R)-4-isopropyl-3-(3-methyl-heptanoyl)-oxazolidin-2-one, (R,R)-3-(3-methyl-pentanoyl)-4-phenyl-oxazolidin-2-one, (R,R)-3-(3-methyl-hexanoyl)-4-phenyl-oxazolidin-2-one, (R,R)-3-(3-methyl-heptanoyl)-4-phenyl-oxazolidin-2-one, (R,R)-4-benzyl-3-(3-methyl-pentanoyl)-oxazolidin-2-one, (R,R)-4-benzyl-3-(3-methyl-hexanoyl)-oxazolidin-2-one, (R,R)-4-benzyl-3-(3-methyl-heptanoyl)-oxazolidin-2-one, (3R,4R,5S)-4-methyl-3-(3-methyl-pentanoyl)-5-phenyl-oxazolidin-2-one, (3R,4R,5S)-4-methyl-3-(3-methyl-hexanoyl)-5-phenyl-oxazolidin-2-one, and (3R,4R,5S)-4-methyl-3-(3-methyl-heptanoyl)-5-phenyl-oxazolidin-2-one.

Scheme V illustrates another method for preparing the β-dicarbonyl (Formula 6) used in Scheme I, II, and III. The method includes activating a chiral alcohol (Formula 34) via, e.g., reaction with a sulfonylating agent (Formula 26), to give an intermediate (Formula 32), which is subsequently reacted with a deprotonated acetoacetate derivative (Formula 36). The resulting chiral anion (Formula 35) or a corresponding salt is treated with an acid to yield, via tautomerization, the desired β-dicarbonyl (Formula 6). As shown in Scheme V, the displacement of substituent R¹⁸ (Formula 32) results in inversion of the stereogenic center and occurs via attack by a dianion intermediate (Formula 36 or corresponding salt). The dianion (Formula 36) may be prepared by treating an acetoacetate derivative (Formula 33) successively with one or more equivalents of a first base (e.g., LiH, NaH, etc.) and a second base (e.g., BuLi) that are strong enough to deprotonate, respectively, the central methylene and terminal methyl groups. Alternatively, the acetoacetate derivative may be treated with two or more equivalents of a single base that can deprotonate the terminal methyl group. In Scheme V, substituents R¹, R², and R³ in Formula 32 and 34 are as defined above in Formula 1; R⁴ in Formula 33 and 36 is as defined above in Formula 2; and R¹⁸ is a leaving group.

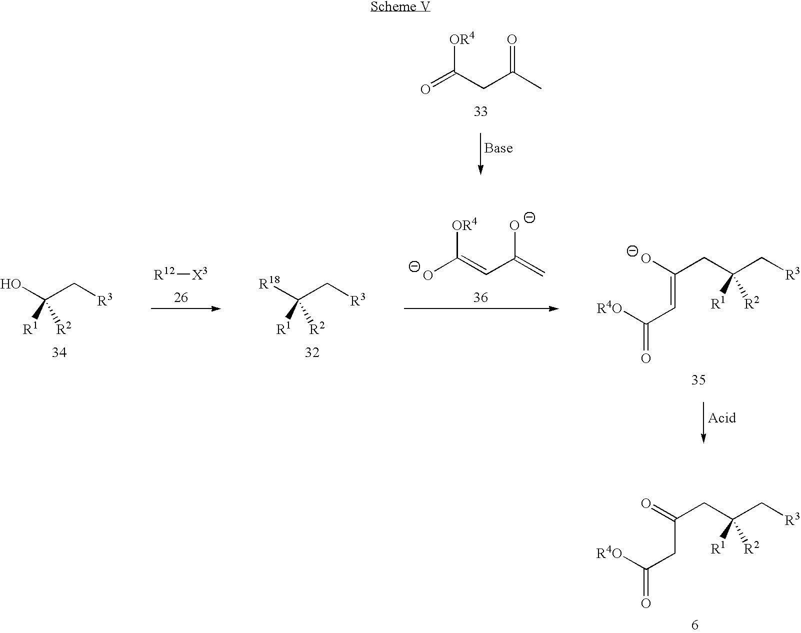

For the methodology shown in Scheme V, representative chiral alcohols (Formula 34) and corresponding activated forms (Formula 32) include, without limitation, (S)-butan-2-ol, (S)-pentan-2-ol, (S)-hex-2-ol; (S)-2-(toluene-4-sulfonyloxy)-butane, (S)-2-(toluene-4-sulfonyloxy)-pentane, (S)-2-(toluene-4-sulfonyloxy)-hexane, (S)-2-(chlorobenzene-4-sulfonyloxy)-butane, (S)-2-(chlorobenzene-4-sulfonyloxy)-pentane, (S)-2-(chlorobenzene-4-sulfonyloxy)-hexane, (S)-2-methanesulfonyloxy-butane, (S)-2-methanesulfonyloxy-pentane, and (S)-2-methanesulfonyloxy-hexane. Representative acetoacetate derivatives (formula 33) include, without limitation, C₁₋₆ alkyl esters of acetoacetate, including acetoacetate ethyl ester. Representative dianions (Formula 36) include, without limitation, Z- and E-isomers of 1-C₁₋₆ alkoxy-buta-1,3-diene-1,3-diol dianions, such as (Z)- and (E)-1-ethoxy-buta-1,3-diene-1,3-diol dianion. Similarly, representative chiral anions (Formula 35) include, without limitation, Z- and E-isomers of (R)-1-C₁₋₆ alkoxy-4-methyl-hex-1-en-2-ol anion, (R)-1-C₁₋₆ alkoxy-4-methyl-hept-1-en-2-ol anion, and (R)-1-C₁₋₆ alkoxy-4-methyl-oct-1-en-2-ol anion, which include Z- and E-isomers of (R)-1-ethoxycarbonyl-4-methyl-hex-1-en-2-ol anion, (R)-1-ethoxycarbonyl-4-methyl-hept-1-en-2-ol anion, and (R)-1-ethoxycarbonyl-4-methyl-oct-1-en-2-ol anion.

The activation of the chiral alcohol (Formula 34), subsequent displacement of R¹⁸ (Formula 32), and treatment of the chiral anion (Formula 35) with acid may be carried out at about −50° C. to reflux, while the preparation of the dianion (Formula 36) generally occurs at temperatures less than about 0° C., and more typically, at temperatures less than about −30° C. but greater than about −80° C.

Many of the compounds described herein are capable of forming pharmaceutically acceptable salts. These salts include, without limitation, acid addition salts (including di-acids) and base salts. Pharmaceutically acceptable acid addition salts include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like.

Pharmaceutically acceptable base salts include nontoxic salts derived from bases, including metal cations, such as an alkali or alkaline earth metal cation, as well as amines. Examples of suitable metal cations include, without limitation, sodium cations (Na⁺), potassium cations (K⁺), magnesium cations (Mg²⁺), calcium cations (Ca²⁺), and the like. Examples of suitable amines include, without limitation, N,N′-dibenzylethylenediamine, chloroprocaine, choline, diethanolamine, dicyclohexylamine, ethylenediamine, N-methylglucamine, and procaine. For a discussion of useful acid addition and base salts, see S. M. Berge et al., “Pharmaceutical Salts,” 66 J. of Pharm. Sci., 1-19 (1977); see also Stahl and Wermuth, Handbook of Pharmaceutical Salts: Properties, Selection, and Use (2002).

One may prepare an acid addition salt (or base salt) by contacting a compound's free base (or free acid) with a sufficient amount of a desired acid (or base) to produce a nontoxic salt. One may then isolate the salt by filtration if it precipitates from solution, or by evaporation to recover the salt. One may also regenerate the free base (or free acid) by contacting the acid addition salt with a base (or the base salt with an acid). Certain physical properties (e.g., solubility, crystal structure, hygroscopicity, etc.) of a compound's free base, free acid, or zwitterion may differ from its acid or base addition salt. Generally, however, references to the free acid, free base or zwitterion of a compound would include its acid and base addition salts.

Disclosed and claimed compounds may exist in both unsolvated and solvated forms and as other types of complexes besides salts. Useful complexes include clathrates or compound-host inclusion complexes where the compound and host are present in stoichiometric or non-stoichiometric amounts. Useful complexes may also contain two or more organic, inorganic, or organic and inorganic components in stoichiometric or non-stoichiometric amounts. The resulting complexes may be ionized, partially ionized, or non-ionized. For a review of such complexes, see J. K. Haleblian, J. Pharm. Sci. 64(8):1269-88 (1975). Pharmaceutically acceptable solvates also include hydrates and solvates in which the crystallization solvent may be isotopically substituted, e.g. D₂O, d₆-acetone, d₆-DMSO, etc. Generally, for the purposes of this disclosure, references to an unsolvated form of a compound also include the corresponding solvated or hydrated form of the compound.

The disclosed compounds also include all pharmaceutically acceptable isotopic variations, in which at least one atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³¹P and ³²P; isotopes of sulfur, such as ³⁵S; isotopes of fluorine, such as ¹⁸F; and isotopes of chlorine, such as ³⁶Cl. Use of isotopic variations (e.g., deuterium, 2H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies.

### EXAMPLES

The following examples are intended as illustrative and non-limiting, and represent specific embodiments of the present invention.

### Example 1

### Preparation of (R)-3-methyl-hexanoic acid from (R)-2-methyl-pentan-1-ol via (R)-3-methyl-hexanenitrile

A mixture of (R)-2-methyl-pentan-1-ol , MeCl₂ and pyridine is cooled to 0° C. to 10° C. To this mixture is added toluenesulfonyl chloride and the resulting reaction mixture is allowed to warm to RT overnight. The reaction is quenched by the addition of water and the mixture is separated into upper and lower layers. The lower layer is washed with dilute aq HCl. The organic layer is concentrated to an oil by vacuum distillation, then diluted with DMSO and reacted by adding sodium cyanide and heating to 50° C. for 3 hours. After cooling to RT, the reaction mixture is diluted with hexane and water and the upper layer is washed with water. The hexane layer is concentrated by vacuum distillation giving (R)-3-methyl-hexanenitrile as an oil. The oil is reacted by adding aq HCl and heating the mixture to 50° C. to 60° C. for 6 hours. The reaction mixture is extracted with diethyl ether and the organic layer is concentrated by vacuum distillation, which provides the titled compound as an oil.

### Example 2

### Preparation of (R)-5-methyl-3-oxo-octanoic acid ethyl ester from (R)-methyl-hexanoic acid and potassium ethyl malonate

To a stirred mixture of N,N carbonyldiimidazole (26.4 g) in THF (175 mL) was added (R)-3-methyl-hexanoic acid (20 g) at RT. The reaction mixture cleared upon stirring for 4 hours at RT to give an activated carboxylic acid solution. To a stirred mixture of ethyl malonate potassium salt (49.6 g) in acetonitrile (265 mL) was added Et₃N (21.4 mL). To this mixture was added anhydrous magnesium chloride powder (35.1 g) in portions while keeping the temperature at 15° C. to 25° C. The resulting slurry was allowed to warm to RT and was stirred for 4 hours. The activated carboxylic acid solution was added to the slurry and the mixture was stirred at RT for 17 hours. The reaction was quenched with aqueous HCl and extracted with MTBE. The organic layer was washed with sodium bicarbonate and NaCl/H₂O/HCl solutions. The solvents were removed by vacuum distillation to give the titled compound as a yellow oil (36 g). ¹H NMR (400 MHz, CDCl₃) δ 4.20 (q, 2H), 3.42 (S, 2H), 2.52 (dd, 1H), 2.33 (dd, 1H), 2.03 (m, 1H), 1.3 (m, 3H), 1.28 (t, 3H), 1.16 (m, 1H), 0.9 (m, 6H).

### Example 3

### Preparation of (R)-5-methyl-3-oxo-octanoic acid ethyl ester from (R)-methyl-hexanoic acid and potassium ethyl malonate

To a stirred mixture of N,N carbonyldiimidazole (33 kg) in EtOAc (217 L) was added (R)-3-methyl-hexanoic acid (25 kg) dissolved in EtOAc (30 L) at RT. The reaction mixture cleared upon stirring for 1 hour at RT to give an activated carboxylic acid solution. To a stirred mixture of ethyl malonate potassium salt (45.8 kg) and magnesium chloride (25.6 kg) in EtOAc (320 L) was added Et₃N (31.1 kg). The activated carboxylic acid solution was added to this slurry and the resulting mixture was stirred at 40° C. to 50° C. for 11 hours. The mixture was cooled to 10° C. to 15° C., and the reaction was quenched with aqueous HCl. The organic layer was washed successively with water and aq sodium bicarbonate and then concentrated by vacuum distillation to give the titled compound as a yellow oil (38.2 kg, 100% yield of 92% pure product).

### Example 4

### Preparation of (R)-5-methyl-3-oxo-octanoic acid ethyl ester from (R,R)-4-methyl-3-(3-methyl-hexanoyl)-5-phenyl-oxazolidin-2-one and potassium ethyl malonate

To a stirred mixture containing (R,R)-4-methyl-3-(3-methyl-hexanoyl)-5-phenyl-oxazolidin-2-one (34 g) and ethyl malonate potassium salt (40 g) in acetonitrile (250 mL), was added magnesium chloride (45 g) in portions while keeping the temperature below 60° C. The slurry was diluted with Et₃N (35 mL) and heated to 60° C. for 16 hours. After cooling to RT, the reaction was quenched with aq HCl and extracted with ethyl acetate. The organic layer was washed with aq sodium bicarbonate and with water. The solvents were removed by vacuum distillation, and the resulting solids were slurried in hexanes (300 mL) and filtered. The solids were 90% pure recovered (4R,5S)-4-methyl-5-phenyl-oxazolidin-2-one. The filtrate was concentrated by vacuum distillation resulting in the titled compound (14 g, 94% pure via vapor phase chromatography). The product could be further purified by vacuum distillation.

### Example 5

### Preparation of (R)-5-methyl-3-oxo-octanoic acid ethyl ester from (R,R)-3-(3-methyl-hexanoyl)-4-phenyl-oxazolidin-2-one and potassium ethyl malonate

To a stirred mixture containing (R,R)-3-(3-methyl-hexanoyl)-4-phenyl-oxazolidin-2-one (5 g) and ethyl malonate potassium salt (6 g) in acetonitrile (50 mL), was added anhydrous magnesium chloride (7.8 g) in portions while keeping the temperature below 60° C. The slurry was diluted with Et₃N (5 mL) and heated to 60° C. for 16 hours. After cooling to RT, the reaction was quenched with aqueous HCl and the upper layer concentrated to an oil, then extracted with hexane. The solvent was removed by vacuum distillation to yield titled compound as an oil (2.3 g). The product could be further purified by vacuum distillation.

### Example 6

### Preparation of (R)-5-methyl-3-oxo-octanoic acid ethyl ester from (S)-pentan-2-ol and acetoacetate ethyl ester

To a 250 mL round bottom flask containing 4-chlorobenzenesulfonyl chloride (25.18 g, 0.12 mol) and (S)-pentan-2-ol (10 g, 0.11 mol) in MeCl₂ (50 mL) was added Et₃N (22.14 mL, 0.15 mol) and DMAP (0.69 g, 0.0057 mol). The mixture was stirred at 40° C. for at least 3 hours. After the reaction was completed, 37% HCl (4.7 mL) and water (10 mL) were added to the reaction mixture. The organic layer was separated and washed with water (25 mL×2). The solvent was removed by distillation at ambient pressure. THF (10 mL×2) was added to remove residual MeCl₂ under vacuum to give (S)-2-(chlorobenzene-4-sulfonyloxy)-pentane as an oil.

To a 1 L round bottom flask containing diisopropylamine (46.7 g, 0.45 mol) in THF (50 mL), which was cooled to −20° C., was slowly added n-BuLi (138.9 g, 0.45 mol) while maintaining the temperature below −10° C. The mixture was cooled to −40° C. After the addition was completed, acetoacetate ethyl ester (29.5 g, 0.22 mol) was slowly added while maintaining the temperature below −30° C. to give 1-ethoxy-buta-1,3-diene-1,3-diol dianion.

To the ethyl acetoacetate dianion was added the pentyl closylate oil (30 g, 0.11 mol) in a single addition. The mixture was allowed to warm to 25° C. and was stirred for at least 3 hours. Following completion of the reaction, an HCl solution (89.4 g in 200 mL water) was added to the cold reaction mixture at 0° C. to quench the reaction. After separating the organic layer, the aqueous layer was extracted with hexane (50 mL). The combined organic layers were washed with NaHCO₃ solution (10 g in 100 mL water) and 10% brine (10 g NaCl and 3 mL of 37% HCl in 100 mL water). The solvent was removed by vacuum distillation. The titled compound was distilled under vacuum at 40° C. to 45° C. (26% yield).

### Example 7

### Preparation (R)-3-methyl-heptanoic acid from (R,R)-3-(3-methyl-heptanoyl)-4-phenyl-oxazolidin-2-one

To a solution of (R,R)-3-(3-methyl-heptanoyl)-4-phenyl-oxazolidin-2-one (24.6 kg) in THF (180 L) and water (30 L), was added lithium hydroperoxide, which was prepared by combining aq LiOH (6.6 kg of LiOH monohydrate dissolved in 130 L of water) and 35% aq hydrogen peroxide (33 kg) at 5±5° C. After stirring for at least 4 hours, the reaction was quenched by the addition of aq sodium bisulfite (42 kg NaHSO₃ dissolved in 140 L water) and was diluted with ethyl acetate (150 kg). The layers were separated and the lower layer was extracted with ethyl acetate (60 kg). The ethyl acetate layers were combined and washed with brine and then concentrated by vacuum distillation. The residue was dissolved in hexanes (280 L) and cooled to crystallize out (R)-4-phenyl-oxazolidin-2-one, which was collected by filtration. The filtrate was concentrated by vacuum distillation to give the titled compound, which was carried directly into the next reaction.

### Example 8

### Preparation of (R)-5-methyl-3-oxo-nonanoic acid ethyl ester from (R)-3-methyl-heptanoic acid and potassium ethyl malonate

To a stirred mixture of CDI (13.2 g) in ethyl acetate (50 mL) was added (R)-3-methyl-heptanoic acid (11.1 g). The mixture was allowed to stir for 4 hours at RT to give an activated acid solution. To a stirred mixture of ethyl malonate potassium salt (18.3 g) in ethyl acetate (125 mL) was added Et₃N (12.4 g). To this mixture was added anhydrous magnesium chloride powder (10.3 g) in portions while keeping the temperature at 15° C. to 25° C. The slurry was allowed to warm to RT and continued to stir for 1 hour. The activated acid solution was subsequently added to the slurry and the mixture was stirred at RT for 17 hours. The reaction was quenched with aqueous HCl. The organic layer was washed with sodium bicarbonate and brine solutions and the solvents were removed by vacuum distillation to yield the titled compound as a yellow oil (15.9 g, 96% yield). ¹H NMR (400 MHz, CDCl₃) δ 4.20 (q, 2H), 3.42 (S, 2H), 2.52 (dd, 1H), 2.33 (dd, 1H), 2.03 (m, 1H), 1.3-1.1 (m, 6H), 1.28 (t, 3H), 0.9 (m, 6H).

### Example 9

### Preparation of (R,E)-5-methyl-oct-2-eneoic acid ethyl ester from (R)-5-methyl-3-oxo-octanoic acid ethyl ester

To a reactor containing (R)-5-methyl-3-oxo-octanoic acid ethyl ester (15 kg, 75 mol) in EtOH (90 kg) was added dichloro-tris(triphenylphosphine)-ruthenium (190 g) followed by 10% aq HCl (0.7 kg). The reactor contents were heated to 50±5° C. and reacted under 50 psig of hydrogen for 20 hours. The reactor was subsequently purged with nitrogen and its contents filtered and concentrated to an oil by vacuum distillation. The oil was diluted with ethyl acetate (60 L), concentrated by vacuum distillation, again diluted with EtOAc (60 L), cooled to −10° C. to −20 C and further diluted with methanesulfonyl chloride (12.1 kg). The resulting solution was cooled to −10° C. to −20 C and Et₃N (26 kg) was slowly added while maintaining the temperature below 20° C. The solution was warmed to 40° C. to 60° C. for at least 12 hours, then cooled to 0° C. to 10° C., and quenched by the addition of aq HCl. The organic solution was washed with water and concentrated by vacuum distillation resulting in an oil. Hexane was added and the solution concentrated by vacuum distillation to give the titled compound (11 kg, 80% yield). ¹H NMR (400 MHz, CDCl₃) δ 6.94 (dt, 1H), 5.80 (d, 1H); 4.18 (q, 2H), 2.19 (m, 1H), 2.05 (m, 1H), 1.63 (m, 1H), 1.3-1.1 (m, 4H), 1.29 (t, 3H), 0.9 (m, 6H).

### Example 10

### Preparation of (1S,3S,5R)-3-[benzyl-(1-phenyl-ethyl)-amino]-5-methyl-octanoic acid ethyl ester from (R)-5-methyl-oct-2-eneoic acid ethyl ester

To a cooled solution of (S)-benzyl-(1-phenyl-ethyl)-amine (21.3 kg) in THF (77 L) was added 24% n-BuLi (27 kg) at −20° C. to −30° C. The resulting solution was cooled to −70° C. to −90° C. and (R,E)-5-methyl-oct-2-eneoic acid ethyl ester (15 kg) in THF (10 L) was slowly added over about 1 hour. The reaction mixture was stirred for an additional 5 to 15 minutes at −65° C. to −75° C. The reaction was subsequently quenched by the addition of aq HCl and the mixture partitioned into upper and lower layers. The upper layer was washed two times with water and the organic layer was concentrated by vacuum distillation to give the titled compound as an oil (30 kg, 93% yield).

### Example 11

### Preparation of (3S,5R)-3-amino-5-methyl-octanoic acid HCl from (1S,3S,5R)-3-[benzyl-(1-phenyl-ethyl)-amino]-5-methyl-octanoic acid ethyl ester

A slurry of 20% Pd/C (10 kg, 50% water wet), (1S,3S,5R)-3-[benzyl-(1-phenyl-ethyl)-amino]-5-methyl-octanoic acid ethyl ester (31 kg) in EtOH (100 L), and acetic acid (10 kg), was reacted with hydrogen (50 psig) at 45° C. to 55° C. for at least 16 hours. Following reaction, the reactor was vented and purged with nitrogen and the contents were filtered. The filtrate was diluted with aq HCl, concentrated by vacuum distillation, and diluted with 35% HCl (30 kg) and water (100 kg). The resulting solution was heated to 80° C. to 100° C. for a minimum of 12 hours and admixed with toluene. The solution was partitioned into upper and lower layers. The lower layer was separated and concentrated by vacuum distillation to a volume of about 50 L. The solution was cooled and the resulting precipitate was collected by filtration, washed with toluene, then dried under vacuum to provide an off-white solid (7 kg). The solid was recrystallized from isopropanol and toluene to give the titled compound as a white product (5 kg, 30% yield). ¹H NMR (400 MHz, D₆DMSO) δ 12.71 (bs, 1H) 8.16 (bs, 3H), 3.38 (m, 1 μl), 2.68 (dd, 1H), 2.53 (dd, 1H), 1.61 (m, 2H), 1.3-1.1 (m, 5H), 0.83 (m, 6H).

### Example 12

### Preparation of (R,E)-5-methyl-non-2-eneoic acid ethyl ester from (R)-5-methyl-3-oxo-nonanoic acid ethyl ester

To a reactor containing (R)-5-methyl-3-oxo-nonanoic acid ethyl ester (16 kg, 75 mol) in EtOH (90 kg) was added dichloro-tris(triphenylphosphine)-ruthenium (96 g) followed by 10% aq HCl (0.7 kg). The contents of the reactor were heated to 50±5° C. under 50 psig of hydrogen for 20 hours. Following reaction, the reactor was purged with nitrogen and the contents were filtered and concentrated to an oil by vacuum distillation. The oil was diluted with ethyl acetate (60 L), concentrated by vacuum distillation, diluted again with EtOAc (60 L), cooled to −10 to −20 C, and further diluted with methanesulfonyl chloride (12.1 kg). The solution was cooled to −10° C. to −20° C. and Et₃N (23 kg) was slowly added while maintaining the temperature below 20° C. The solution was warmed to about 50° C. for at least 12 hours, cooled to 0° C. to 10° C., and quenched with aq HCl. The organic solution was washed with water and concentrated by vacuum distillation resulting in an oil. Hexane was added and the solution concentrated by vacuum distillation to give the titled compound (11 kg, 75% yield). ¹H NMR (400 MHz, CDCl₃) δ 6.94 (dt, 1H), 5.80 (d, 1H), 4.18 (q, 2H), 2.20 (m, 1H), 2.05 (m, 1H), 1.60 (m, 1H), 1.3-1.1 (m, 6H), 1.29 (t, 3H), 0.9 (m, 6H).

### Example 13

### Preparation of (1S,3S,5R)-3-[benzyl-(1-phenyl-ethyl)-amino]-5-methyl-nonanoic acid ethyl ester from (R)-5-methyl-non-2-eneoic acid ethyl ester

To a cooled solution of (S)-benzyl-(1-phenyl-ethyl)-amine (29 kg) in THF (250 L) was added 24% n-BiLi (34.5 kg) at −20° C. to −30° C. The resulting solution was cooled to −70° C. to −90° C. and (R)-5-methyl-non-2-eneoic acid ethyl ester (19.5 kg) in THF (70 L) was slowly added over about 1 hour. The reaction mixture was stirred for an additional 5 to 15 minutes at −65° C. to −75° C. The reaction was quenched by the addition of aqueous HCl and the mixture was partitioned between upper and lower layers. The upper layer was washed two times with water and the organic layer was concentrated by vacuum distillation to give the titled compound as an oil (32 kg, 79% yield).

### Example 14

### Preparation of (3S,5R)-3-amino-5-methyl-nonanoic acid HCl from (1S,3S,5R)-3-[benzyl-(1-phenyl-ethyl)-amino]-5-methyl-nonanoic acid ethyl ester

A slurry of 20% Pd/C (20 kg, 50% water wet), (1S,3S,5R)-3-[benzyl-(1-phenyl-ethyl)-amino]-5-methyl-nonanoic acid ethyl ester (50 kg) in EtOH (312 L), and acetic acid (15 kg) acetic acid, was reacted with hydrogen (50 psig) at 45° C. to 55° C. for at least 16 hours. Following reaction, the reactor was vented and purged with nitrogen and the contents were filtered. The filtrate was diluted with aq HCl, concentrated by vacuum distillation, and diluted with 50 Kg 35% HCl (50 kg) and 100 Kg water (100 kg). The solution was heated to 80° C. to 100° C. for a minimum of 12 hours and then admixed with toluene. The solution was partitioned into upper and lower layers. The lower layer was separated and concentrated by vacuum distillation to a volume of about 100 L. The solution was diluted with concentrated aq. HCl (12 kg) and cooled. The resulting precipitate was collected by filtration, washed with toluene, then dried under vacuum to give an off-white solid (21 kg). The solid was recrystallized twice from aq HCl to provide the titled compound as a white solid (11 kg, 40% yield). ¹H NMR (400 MHz, D₆DMSO) δ 12.77 (bs, 1H), 8.14 (bs, 3H), 3.36 (m, 1H), 2.67 (dd, 1H), 2.53 (dd, 1H), 1.60 (m, 2H), 1.3-1.1 (m, 7H), 0.83 (m, 6H).

### Example 15

### Preparation of (3S,5R)-3-amino-5-methyl-nonanoic acid from (3S,5R)-3-amino-5-methyl-nonanoic acid HCl

(3S,5R)-3-amino-5-methyl-nonanoic acid HCl (11 kg) was dissolved in water (45 L), filtered to remove particulates, and diluted with aq 5 N NaOH (10 L) to give a slurry having a pH between 5 and 7. The solids were dissolved by MTBE (50 L) and the solution cooled to −5° C. to 5 C. The resulting precipitate was collected by filtration and washed with cooled MTBE. The solids were slurried in water (about 50 L), collected by filtration, washed with MTBE, then dried in a vacuum oven to give the titled compound as a white solid (3.8 kg, 42% yield). ¹H NMR (400 MHz, CD₃OD) δ 4.93 (bs, 2H), 3.42 (m, 1H), 2.46 (dd, 1H), 2.28 (dd, 1H), 1.61 (m, 2H), 1.5-1.1 (m, 7H), 0.9 (m, 6H).

### Example 16

### Preparation of (R,Z)-3-acetylamino-5-methyl-oct-2-enoic acid ethyl ester from (R)-5-methyl-3-oxo-octanoic acid ethyl ester

(R)-5-Methyl-3-oxo-octanoic acid ethyl ester (20 g) and ammonium acetate (16.2 g) were heated in EtOH (150 mL) at 60° C. for 3 hours, then cooled and concentrated to an oil by vacuum distillation. The material was dissolved in toluene and concentrated by vacuum distillation. After adding toluene and cooling to 15° C. to 25° C., the slurry was filtered, diluted with additional toluene, and reacted further by adding acetic anhydride (20 g) and pyridine (21.3 mL) and heating the mixture at 100° C. to 110° C. for 16 hours. The reaction mixture was cooled to 10° C. to 20° C. and the reaction was quenched by the addition of water. The mixture was partitioned into upper and lower layers, and the upper layer was washed with dilute aqueous sulfuric acid followed by water. The product was concentrated by vacuum distillation, dissolved in EtOH, and concentrated again to give the titled compound as an oil (21 g, 88% yield). ¹H NMR (CDCl₃) δ 4.90 (s, 1H), 4.17 (q, J=8 Hz, 2H), 2.88 (m, 1H), 2.34 (m, 1H), 2.14 (s, 3H), 1.79 (m, 1H), 1.30 (m, 7H), 0.89 (m, 6H).

### Example 17

### Preparation of (3S,5R)-3-acetylamino-5-methyl-octanoic acid ethyl ester via asymmetric hydrogenation of (R,Z)-3-acetylamino-5-methyl-oct-2-enoic acid ethyl ester using (R,R,S,S)-TangPhos-Rh catalyst

A 250 mL 3NRB glass flask fitted with a magnetic stirrer, a rubber septum, a glass stopper, and a gas inlet adapter, was purged by evacuating and filling the flask with nitrogen four times. The flask was then placed in a nitrogen-filled glove bag along with a vial containing bis(1,5-cyclooctadiene)Rh(I)trifluoromethane sulfonate (0.123 g, 0.263 mmol, 1.00 mol %) and a vial containing (R,R,S,S)-TangPhos (0.070 g, 0.245 mmol, 0.93 mol %). The vials were opened and their contents were charged to the flask. The flask, which contained the catalyst precursors, was sealed, moved to a hood, purged again with vacuum and nitrogen, and held under a positive nitrogen pressure.

To a separate 250 mL 3NRB glass flask fitted with a magnetic stir bar was charged (R,Z)-3-acetylamino-5-methyl-oct-2-enoic acid ethyl ester (6.33 g, 26.2 mmol) and MeOH (120 mL). This flask was sealed with a rubber septum, a glass stopper, and a gas inlet adapter with a PTFE stopcock. While stirring its contents, the flask was purged four times by evacuating and filing the flask with nitrogen. The flask was then held under a positive nitrogen pressure and its contents were transferred, via syringe, to the reaction flask, which contained the catalyst precursors.

The reaction flask was again inerted (with agitation) using several vacuum/nitrogen purges. Hydrogen was then introduced as a rapid stream that was vented through a bubbler. After about 10 minutes the hydrogen flow rate was reduced so that it maintained a small positive pressure, estimated at about 5 psig to about 10 psig, as indicated by the bubbler. The reaction was run at ambient temperature, without heating or cooling to give the titled compound. Samples were taken via syringe for TLC and chiral GC analysis, and the reaction was found to be complete after about 4 hours (97.1% de via chiral GC). ¹H NMR (D₆DMSO) δ 7.66 (d, J=8 Hz, 1H), 4.13 (m, 1H), 4.01 (q, J=7 Hz, 2H), 2.34 (m, 2H), 1.75 (s, 3H), 1.40 (m, 2M), 1-1.3 (m, 8H), 0.8 (m, 6H).

### Example 18

### Preparation of (3S,5R)-3-acetylamino-5-methyl-octanoic acid ethyl ester via asymmetric hydrogenation of (R,Z)-3-acetylamino-5-methyl-oct-2-enoic acid ethyl ester using (R)-BINAPINE-Rh catalyst

(R,Z)-3-Acetylamino-5-methyl-oct-2-enoic acid ethyl ester (5 g) was dissolved in methanol (15 mL) and the solution thoroughly degassed and inerted with argon. (R)-BINAPINE-Rh(COD)BF₄ (30 mg) was added in a glove box, and the vessel sealed and placed under hydrogen (75 psig). The reaction was stirred vigorously and warmed to 30° C. Following completion of the reaction, the reaction mixture was concentrated to give the titled compound as an oil (5 g, 96% de).

### Example 19

### Preparation of (3S,5R)-3-acetylamino-5-methyl-octanoic acid ethyl ester via asymmetric hydrogenation of (R,Z)-3-acetylamino-5-methyl-oct-2-enoic acid ethyl ester using (R)-mTCFP-Rh catalyst

(R,Z)-3-Acetylamino-5-methyl-oct-2-enoic acid ethyl ester (52 g) was dissolved in methanol (150 mL) and the solution thoroughly degassed and inerted with argon. (R)-mTCFP-Rh(COD)BF₄ (178 mg) was added in a glove box, and the vessel sealed and placed under hydrogen (10 psig). The reaction was stirred vigorously and warmed to 35° C. Following completion of the reaction, the reaction mixture was concentrated to give the titled compound as an oil (52 g, 93.2% de). ¹H NMR (CDCl₃) δ 5.98 (d, 1H), 4.35 (m, 1H), 4.15 (q, 2H), 2.47 (m, 2H), 1.97 (s, 3H), 1.57 (m, 1H), 1-1.4 (m, 8H), 0.9 (m, 6H).

### Example 20

### Preparation of (3S,5R)-3-amino-5-methyl-octanoic acid HCl from (3S,5R)-3-acetylamino-5-methyl-octanoic acid ethyl ester

(3S,5R)-3-Acetylamino-5-methyl-octanoic acid ethyl ester (4.5 g) was diluted with 35% aq HCl (4 mL) and water (8 mL) and heated to 95° C. to 105° C. for 3 days. Toluene (10 mL) was added to the mixture, which was cooled to 5° C. The product was collected by filtration, and washed with toluene resulting in the titled compound (2.6 g). The product may be further purified by recrystallizing from aqueous HCl or toluene/isopropanol, and then filtered, washed with toluene, and dried under vacuum. ¹H NMR (DDMSO) δ 12.7 (bs, 1H), 8.17 (bs, 3H), 3.38 (m, 1H), 2.69 (dd, J=6, 17 Hz, 1H), 2.53 (dd, J=7, 17 Hz, 1H), 1.61 (m, 2H), 1.2 (m, 4H), 1.1 (m, 1H), 0.8 (m, 6H).

### Example 21

### Preparation of (R,Z)-3-acetylamino-5-methyl-non-2-enoic acid ethyl ester from (R)-5-methyl-3-oxo-nonanoic acid ethyl ester via (R,Z)-3-amino-5-methyl-non-2-enoic acid ethyl ester

(R)-5-Methyl-3-oxo-nonanoic acid ethyl ester (30 g) and ammonium acetate (22 g) were heated in EtOH (150 mL) at 60° C. for 16 hours, then cooled and concentrated by vacuum distillation. The material was dissolved in toluene and concentrated by vacuum distillation. The resulting concentrate was dissolved in toluene, filtered to remove solids, and concentrated by vacuum distillation to give (R,Z)-3-amino-5-methyl-non-2-enoic acid ethyl ester as an oil (29 g). A portion of the enamine (25 g) was dissolved in toluene (150 mL), and reacted further by adding acetic anhydride (24 g) and pyridine (24 mL) and heating the mixture at 100° C. to 110° C. for 16 hours. The reaction mixture was cooled to 10° C. to 20° C. and quenched by the addition of water. The reaction mixture was partitioned into upper and lower layers and the upper layer was washed with dilute aqueous NaHSO₄, water, and was concentrated to give the titled compound as an oil (26 g). ¹H NMR (CDCl₃) δ 11.2 (s, 1H), 4.90 (s, 1H), 4.17 (q, J=8 Hz, 2H), 2.88 (dd, 1H, J=8, 16 Hz), 2.34 (dd, 1H, J=12, 8 Hz), 2.14 (s, 3H), 1.79 (m, 1H), 1.30 (m, 7H), 0.89 (m, 6H); ¹³C NMR (CDCl₃) 169.1, 1668.3, 157.8, 97.2, 59.8, 42.7, 36.3, 31.4, 29.0, 28.8, 22.9, 22.7, 19.0, 14.2, 14.0.

### Example 22

### Preparation of (R,Z)-3-amino-5-methyl-non-2-enoic acid ethyl ester from (R)-5-methyl-3-oxo-nonanoic acid ethyl ester

A pressure vessel was charged with (R)-5-Methyl-3-oxo-nonanoic acid ethyl ester (50 g), EtOH (250 mL), and ammonia (about 8 g). The resulting mixture was allowed to react at 50° C. for about 20 hours. The mixture was subsequently cooled to RT and concentrated by vacuum distillation. The resulting concentrate was dissolved in octane and concentrated by vacuum distillation to give the titled compound as an oil (50 g).

### Example 23

### Preparation of (3S,5R)-3-acetylamino-5-methyl-nonanoic acid HCl via asymmetric hydrogenation of (R,Z)-3-acetylamino-5-methyl-non-2-enoic acid ethyl ester using (R)-BINAPINE-Rh catalyst

(R,Z)-3-Acetylamino-5-methyl-non-2-enoic acid ethyl ester (0.64 g) and (R)-BINAPINE-Rh(COD)BF₄ (5.2 mg) were dissolved under inert conditions in MeOH (3 mL) and were reacted with hydrogen (30 psig) at 30° C. Following completion of the reaction, the mixture was concentrated to an oil and diluted with 35% aq HCl (0.6 g) and water (0.6 mL) and heated at 100° C. to 105° C. for 50 hours. The solution was cooled to 0° C. to 10° C. and filtered to give the titled compound.

### Example 24

### Preparation of (3S,5R)-3-acetylamino-5-methyl-nonanoic acid ethyl ester via asymmetric hydrogenation of (R,Z)-3-acetylamino-5-methyl-non-2-enoic acid ethyl ester using (R)-BINAPINE-Rh catalyst and (R)-mTCFP-Rh catalyst

A pressure vessel was charged with (R,Z)-3-acetylamino-5-methyl-non-2-enoic acid ethyl ester (100 kg) and MeOH (320 kg) and was purged with nitrogen. (R)-BINAPINE-Rh(COD)BF₄ (500 g) was added and rinsed into the vessel using nitrogen-purged MeOH (20 L). The reaction vessel was purged with hydrogen and the contents allowed to react at 35° C. under 25 psig H₂ for 2 to 5 days. (R)-mTCFP-Rh(COD)BF₄ (about 60 g) was added and rinsed into the vessel using nitrogen-purged MeOH (20 L). The reaction was allowed to continue at 35° C. under 25 psig H₂. Following completion of the reaction, the mixture was concentrated by vacuum distillation to give the titled compound as an oil. ¹H NMR (CDCl₃) δ 5.97 (d, 1H), 4.35 (m, 1H), 4.15 (q, 2H), 2.56 (dd, 1H), 2.47 (dd, 1H), 1.97 (s, 3H), 1.57 (m, 1H), 1.42 (m, 1H), 1.1-1.3 (m, 10H), 0.9 (m, 6H).

### Example 25

### Preparation of (3S,5R)-3-amino-5-methyl-nonanoic acid HCl salt from (3S,5R)-3-acetylamino-5-methyl-nonanoic acid ethyl ester

(3S,5R)-3-Acetylamino-5-methyl-nonanoic acid ethyl ester (150 kg) was diluted with 35% aq HCl (150 L) and water (300 L). The resulting mixture was heated and stirred at 100° C. to 115° C. for at least 48 hours while distilling off a portion of the solvent (about 100 L). The solution was cooled to 35° C. to 50° C., washed with toluene (300 L), and concentrated slightly by vacuum distillation. The resulting concentrate was diluted with toluene (300 L). Adding 35% aq HCl (150 L) and slowly cooling the solution to about 10° C. resulted in a solid precipitate, which was collected by filtration and washed with hexane and dried. The solids were dissolved in i-PrOH and were recrystallized by adding hexanes and slowly cooling the solution. The solids were collected by filtration, washed with hexanes, and then dried to give the titled compound as a solid (51 kg, 39% yield). ¹H NMR (400 MHz, D₆DMSO) δ 12.7 (bs, 1H), 8.14 (bs, 3H), 3.36 (m, 1H), 2.67 (dd, 1H), 2.53 (dd, 1H), 1.60 (m, 2H), 1.3-1.1 (m, 7H), 0.84 (m, 6H).

### Example 26

### Preparation of (3S,5R)-3-amino-5-methyl-nonanoic acid (zwitterion) from (3S,5R)-3-amino-5-methyl-nonanoic acid HCl Salt

(3S,5R)-3-Amino-5-methyl-nonanoic acid HCl salt (51 kg) was dissolved in water (170 L), passed through a polish filter, and titrated with aq NaOH until the pH of the solution was 5.5 to 7.5. MTBE (200 L) was added and the resulting mixture was warmed to 25° C. to 35° C. and slowly cooled to 0° C. to 10° C. to form a solid precipitate. The precipitate was collected by filtration, washed successively with a small amount of water and with MTBE and then dried to give the titled compound as a white solid (41 kg, 95% yield). ¹H NMR (400 MHz, CD3OD) δ 4.93 (bs, 2H), 3.42 (m, 1H), 2.46 (dd, 1H), 2.28 (dd, 1H), 1.61 (m, 2H), 1.5-1.1 (m, 7H), 0.9 (m, 6H).

### Example 27

### Preparation of (3S,5R)-3-amino-5-methyl-nonanoic acid HCl salt from (3S,5R)-3-acetylamino-5-methyl-nonanoic acid ethyl ester via (3S,5R)-3-acetylamino-5-methyl-nonanoic acid

To a solution containing (3S,5R)-3-acetylamino-5-methyl-nonanoic acid ethyl ester in MeOH is added aq NaOH. The resulting mixture is stirred for 2 hours or until complete to give (3S,5R)-3-acetylamino-5-methyl-nonanoic acid sodium salt. The amide acid salt is concentrated by vacuum distillation to about one-third of its volume. Water and toluene are added to the concentrate and the phases separated. Aq HCl is added to the lower layer and the resulting solution is heated to 100° C. to 110 C for at least 36 hours. The solution is cooled to precipitate a solid, which is collected by filtration and washed with hexane to give the above-titled compound.

### Example 28

### Preparation of (3S,5R)-3-amino-5-methyl-nonanoic acid ethyl ester via asymmetric hydrogenation of (R,Z)-3-amino-5-methyl-non-2-enoic acid ethyl ester

A pressure vessel is charged with (R,Z)-3-amino-5-methyl-non-2-enoic acid ethyl ester (10 g) and 2,2,2-trifluoroethanol (32 g) and the contents are purged with nitrogen. To the vessel is added (R)—(S)-JOSIPHOS-Rh(COD)BF₄ (50 mg). The vessel contents are purged with hydrogen and are reacted at 50° C. under 100 psig H₂ for 1 to 2 days or until the reaction is complete. The mixture is concentrated by vacuum distillation to give the above titled compound.

### Example 29

### Preparation of (3S,5R)-3-amino-5-methyl-nonanoic acid from (3S,5R)-3-amino-5-methyl-nonanoic acid ethyl ester

(3S,5R)-3-Amino-5-methyl-nonanoic acid ethyl ester (10 g) is diluted with 35% aq HCl (10 mL) and water (20 mL) and is heated with stirring at 100° C. to 115° C. for at least 6 hours while distilling off about 2 mL of solvent. The solution is cooled to 35° C. to 50° C. and is washed with toluene (20 mL). The solution is concentrated slightly by vacuum distillation and is diluted with toluene (20 mL). Concentrated aq HCl (10 ml, 35%) is added and the solution is cooled slowly to about 10° C. to precipitate the above titled compound, which is collected by filtration, washed with hexane, and dried. The solid may be recrystallized from i-PrOH and hexanes.

It should be noted that, as used in this specification and the appended claims, singular articles such as “a,” “an,” and “the,” may refer to one object or to a plurality of objects unless the context clearly indicates otherwise. Thus, for example, reference to a composition containing “a compound” may include a single compound or two or more compounds.

It is to be understood that the above description is intended to be illustrative and not restrictive. Many embodiments will be apparent to those of skill in the art upon reading the above description. The scope of the invention should, therefore, be determined not with reference to the appended claims, along with the full scope of equivalents to which such claims are entitled. The disclosures of all articles and references, including patent applications, granted patents, and publications, are incorporated herein by reference in their entirety and for all purposes.

What is claimed is:

1. A method of making a compound of Formula 1, 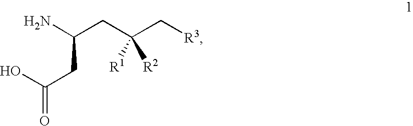
or a diastereomer thereof or a pharmaceutically acceptable complex, salt, solvate or hydrate thereof, wherein
R¹ and R² are independently hydrogen atoms or C₁₋₃ alkyl optionally substituted with one to five fluorine atoms, provided that when R¹ is a hydrogen atom, R² is not a hydrogen atom; and
R³ is C₁₋₆ alkyl, C₃₋₆ cycloalkyl, C₃₋₆ cycloalkyl-C₁₋₆ alkyl, aryl, aryl-C₁₋₃ alkyl, or arylamino, wherein each alkyl of R³ is optionally substituted with one to five fluorine atoms, and each aryl of R³ is optionally substituted with from one to three substituents independently selected from chloro, fluoro, amino, nitro, cyano, C₁₋₃ alkylamino, C₁₋₃ alkyl optionally substituted with one to three fluorine atoms, and C₁₋₃ alkoxy optionally substituted with from one to three fluorine atoms;
the method comprising:
reacting a compound of Formula 2, 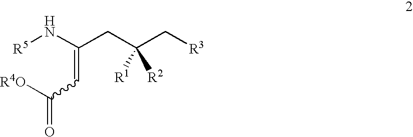
or Formula 4, 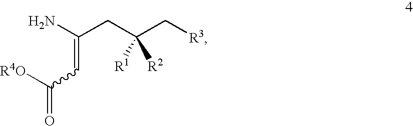
with H₂ in the presence of a chiral catalyst to give a compound of Formula 3, 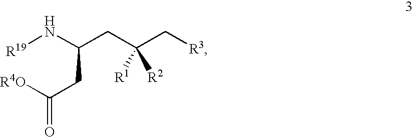
or a diastereomer thereof, wherein R¹, R², and R³ in Formula 2, Formula 3, and Formula 4 are as defined in Formula 1; R⁴ in Formula 2, Formula 3, and Formula 4 is a hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl or a cation selected from a Group 1 metal ion, a Group 2 metal ion, a primary ammonium ion or a secondary ammonium ion; and R⁵ in Formula 2 and R¹⁹ in Formula 3 are independently hydrogen atom, carboxy, C₁₋₇ alkanoyl, C₂₋₇ alkenoyl, C₂₋₇ alkynoyl, C₃₋₇ cycloalkanoyl, C₃₋₇ cycloalkenoyl, halo-C₁₋₇ alkanoyl, halo-C₂₋₇ alkenoyl, halo-C₂₋₇ alkynoyl, C₁₋₆ alkoxycarbonyl, halo-C₁₋₆ alkoxycarbonyl, C₃₋₇ cycloalkoxycarbonyl, aryl-C₁₋₇ alkanoyl, aryl-C₂₋₇ alkenoyl, aryl-C₂₋₇ alkynoyl, aryloxycarbonyl, or aryl-C₁₋₄ alkoxycarbonyl, provided that R⁵ is not a hydrogen atom;
optionally converting the compound of Formula 3 or its diastereomer to the compound of Formula 1 or its diastereomer or to a pharmaceutically acceptable complex, salt, solvate or hydrate of the compound of Formula 1 or its diastereomer.

2. The method of claim 1, wherein the chiral catalyst comprises a chiral ligand bound to a transition metal through one or more phosphorus atoms.

3. The method of claim 2, wherein the chiral ligand is (R,R,S,S)-TANGPhos, (R)-BINAPINE, (R)-eTCFP, or (R)-mTCFP, or stereoisomers thereof.

4. A method of making a compound of Formula 1, 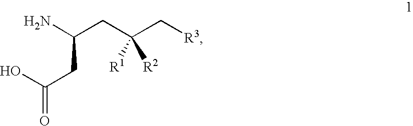
or a diastereomer thereof or a pharmaceutically acceptable complex, salt, solvate or hydrate thereof, wherein
R¹ and R² are independently hydrogen atoms or C₁₋₃ alkyl optionally substituted with one to five fluorine atoms, provided that when R¹ is a hydrogen atom, R² is not a hydrogen atom; and
R³ is C₁₋₆ alkyl, C₃₋₆ cycloalkyl, C₃₋₆ cycloalkyl-C₁₋₆ alkyl, aryl, aryl-C₁₋₃ alkyl, or arylamino, wherein each alkyl of R³ is optionally substituted with one to five fluorine atoms, and each aryl of R³ is optionally substituted with from one to three substituents independently selected from chloro, fluoro, amino, nitro, cyano, C₁₋₃ alkylamino, C₁₋₃ alkyl optionally substituted with one to three fluorine atoms, and C₁₋₃ alkoxy optionally substituted with from one to three fluorine atoms;
the method comprising:
reducing an amino moiety of a compound of Formula 7, 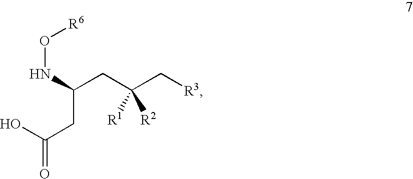
or a diastereomer thereof or a salt thereof to give the compound of Formula 1, wherein R¹, R², and R³ in Formula 7 are as defined in Formula 1 and R⁶ is C₁₋₆ alkyl, C₂₋₆ alkenyl or aryl-C₁₋₃ alkyl; and
optionally converting the compound of Formula 1 or its diastereomer to a pharmaceutically acceptable complex, salt, solvate or hydrate.

5. A method of making a compound of Formula 1, 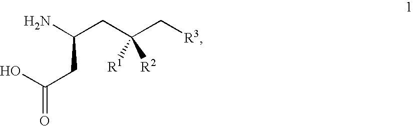
or a diastereomer thereof or a pharmaceutically acceptable complex, salt, solvate or hydrate thereof, wherein
R¹ and R² are independently hydrogen atoms or C₁₋₃ alkyl optionally substituted with one to five fluorine atoms, provided that when R¹ is a hydrogen atom, R² is not a hydrogen atom; and
R³ is C₁₋₆ alkyl, C₃₋₆ cycloalkyl, C₃₋₆ cycloalkyl-C₁₋₆ alkyl, aryl, aryl-C₁₋₃ alkyl, or arylamino, wherein each alkyl of R³ is optionally substituted with one to five fluorine atoms, and each aryl of R³ is optionally substituted with from one to three substituents independently selected from chloro, fluoro, amino, nitro, cyano, C₁₋₃ alkylamino, C₁₋₃ alkyl optionally substituted with one to three fluorine atoms, and C₁₋₃ alkoxy optionally substituted with from one to three fluorine atoms;
the method comprising:
reducing an amine moiety of a compound of Formula 13, 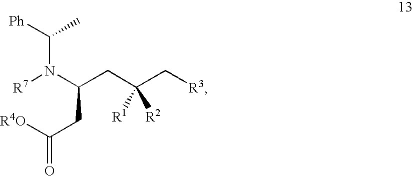
or a diastereomer thereof, to give a compound of Formula 37, 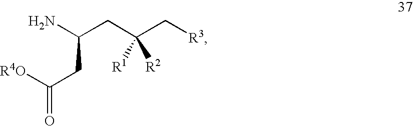
or a diastereomer thereof, wherein R¹, R², and R³ in Formula 37 and Formula 13 are as defined in Formula 1, R⁴ in Formula 37 and Formula 13 is a hydrogen atom, C₁₋₆ alkyl, C₂₋₄ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl or a cation selected from a Group 1 metal ion, a Group 2 metal ion, a primary ammonium ion or a secondary ammonium ion, and R⁷ in Formula 13 is C₁₋₆ alkyl, C₂, alkenyl or aryl-C₁₋₃ alkyl; and
optionally converting the compound of Formula 37 or its diastereomer to the compound of Formula 1 or its diastereomer or to a pharmaceutically acceptable complex, salt, solvate or hydrate of the compound of Formula 1 or its diastereomer.

6. A method of making a compound of Formula 6, 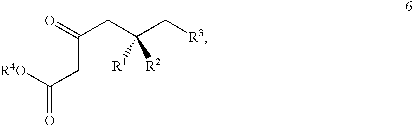
wherein
R¹ and R² are independently hydrogen atoms or C₁₋₃ alkyl optionally substituted with one to five fluorine atoms, provided that when R¹ is a hydrogen atom, R² is not a hydrogen atom; and
R³ is C₁₋₆ alkyl, C₃₋₆ cycloalkyl, C₃₋₆ cycloalkyl-C₁₋₆ alkyl, aryl, aryl-C₁₋₃ alkyl, or arylamino, wherein each alkyl of R³ is optionally substituted with one to five fluorine atoms, and each aryl of R³ is optionally substituted with from one to three substituents independently selected from chloro, fluoro, amino, nitro, cyano, C₁₋₃ alkylamino, C₁₋₃ alkyl optionally substituted with one to three fluorine atoms, and C₁₋₃ alkoxy optionally substituted with from one to three fluorine atoms; and
R⁴ is a hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl or a cation selected from a Group 1 metal ion, a Group 2 metal ion, a primary ammonium ion or a secondary ammonium ion;
the method comprising treating a compound of Formula 19, 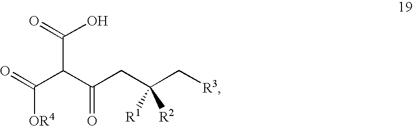
or a salt thereof with an acid, wherein R¹, R², R³, and R⁴ in Formula 19 are as defined in Formula 6.

7. A method of making a compound of Formula 6, 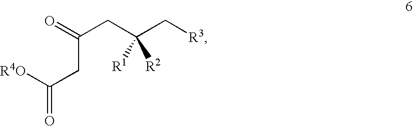
wherein
R¹ and R² are independently hydrogen atoms or C₁₋₃ alkyl optionally substituted with one to five fluorine atoms, provided that when R¹ is a hydrogen atom, R² is not a hydrogen atom; and
R³ is C₁₋₆ alkyl, C₃₋₆ cycloalkyl, C₃₋₆ cycloalkyl-C₁₋₆ alkyl, aryl, aryl-C₁₋₃ alkyl, or arylamino, wherein each alkyl of R³ is optionally substituted with one to five fluorine atoms, and each aryl of R³ is optionally substituted with from one to three substituents independently selected from chloro, fluoro, amino, nitro, cyano, C₁₋₃ alkylamino, C₁₋₃ alkyl optionally substituted with one to three fluorine atoms, and C₁₋₃ alkoxy optionally substituted with from one to three fluorine atoms; and
R⁴ is a hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl or a cation selected from a Group 1 metal ion, a Group 2 metal ion, a primary ammonium ion or a secondary ammonium ion;
the method comprising treating a compound of Formula 33, 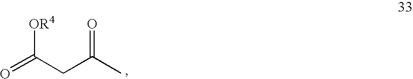
with a base to generate a dianion;
reacting the dianion with a compound of Formula 32, 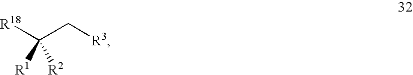
to give an intermediate; and
treating the intermediate with an acid, wherein R¹, R², and R³ in Formula 32 and R⁴ in Formula 33 are as defined in Formula 6 and R¹⁸ in Formula 32 is a leaving group.

8. A compound of Formula 40, 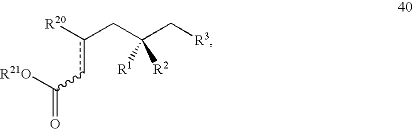
including complexes, salts, solvates, hydrates, opposite enantiomers, diastereomers, geometric isomers, and mixtures thereof, in which:
R¹ and R² are independently hydrogen atoms or C₁₋₃ alkyl optionally substituted with one to five fluorine atoms, provided that when R¹ is a hydrogen atom, R² is not a hydrogen atom;
R³ is C₁₋₆ alkyl, C₃₋₆ cycloalkyl, C₃₋₆ cycloalkyl-C₁₋₆ alkyl, aryl, aryl-C₁₋₃ alkyl, or arylamino, wherein each alkyl of R³ is optionally substituted with one to five fluorine atoms, and each aryl of R³ is optionally substituted with from one to three substituents independently selected from chloro, fluoro, amino, nitro, cyano, C₁₋₃ alkylamino, C₁₋₃ alkyl optionally substituted with one to three fluorine atoms, and C₁₋₃ alkoxy optionally substituted with from one to three fluorine atoms;
R²⁰ is a hydrogen atom, hydroxy, R⁶—O—NH—, R⁹⁰— or R¹⁹—NH—, or 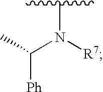
and
R²¹ is a hydrogen atom, C₁₋₆ alkyl, C₂₋₄ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl or a cation selected from a Group 1 metal ion, a Group 2 metal ion, a primary ammonium ion, a secondary ammonium ion or R⁶—O—NH—; wherein:
R⁶ and R⁷ are independently C₁₋₆ alkyl, C₂₋₆ alkenyl or aryl-C₁₋₃ alkyl;
R⁹ is tosyl, mesyl, brosyl, closyl, nosyl, or triflyl; and
R¹⁹ is hydrogen atom, carboxy, C₁₋₇ alkanoyl, C₂₋₇ alkenoyl, C₂₋₇ alkynoyl, C₃₋₇ cycloalkanoyl, C₃₋₇ cycloalkenoyl, halo-C₁₋₇ alkanoyl, halo-C₂₋₇ alkenoyl, halo-C₂₋₇ alkynoyl, C₁₋₆ alkoxycarbonyl, halo-C₁₋₆ alkoxycarbonyl, C₃₋₇ cycloalkoxycarbonyl, aryl-C₁₋₇ alkanoyl, aryl-C₂₋₇ alkenoyl, aryl-C₂₋₇ alkynoyl, aryloxycarbonyl, or aryl-C₁₋₆ alkoxycarbonyl.

9. A compound of Formula 39, 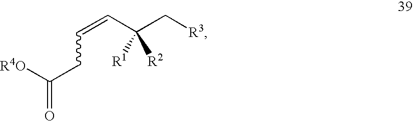
including complexes, salts, solvates, hydrates, opposite enantiomers, diastereomers, geometric isomers, and mixtures thereof, in which:
R¹ and R² are independently hydrogen atoms or C₁₋₃ alkyl optionally substituted with one to five fluorine atoms, provided that when R¹ is a hydrogen atom, R² is not a hydrogen atom;
R³ is C₁₋₄ alkyl, C₃₋₄ cycloalkyl, C₃₋₆ cycloalkyl-C₁₋₆ alkyl, aryl, aryl-C₁₋₃ alkyl, or arylamino, wherein each alkyl of R³ is optionally substituted with one to five fluorine atoms, and each aryl of R³ is optionally substituted with from one to three substituents independently selected from chloro, fluoro, amino, nitro, cyano, C₁₋₃ alkylamino, C₁₋₃ alkyl optionally substituted with one to three fluorine atoms, and C₁₋₃ alkoxy optionally substituted with from one to three fluorine atoms; and
R⁴ is a hydrogen atom, C₁₋₆ allyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl or a cation selected from a Group 1 metal ion, a Group 2 metal ion, a primary ammonium ion or a secondary ammonium ion.

10. A compound of Formula 41, 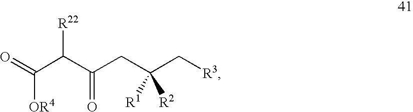
including complexes, salts, solvates, hydrates, opposite enantiomers, diastereomers, geometric isomers, and mixtures thereof, in which:
R¹ and R² are independently hydrogen atoms or C₁₋₃ alkyl optionally substituted with one to five fluorine atoms, provided that when R¹ is a hydrogen atom, R² is not a hydrogen atom;
R³ is C₁₋₆ alkyl, C₃₋₆ cycloalkyl, C₃₋₆ cycloalkyl-C₁₋₆alkyl, aryl, aryl-C₁₋₃ alkyl, or arylamino, wherein each alkyl of R³ is optionally substituted with one to five fluorine atoms, and each aryl of R³ is optionally substituted with from one to three substituents independently selected from chloro, fluoro, amino, nitro, cyano, C₁₋₃ alkylamino, C₁₋₃ alkyl optionally substituted with one to three fluorine atoms, and C₁₋₃ alkoxy optionally substituted with from one to three fluorine atoms;
R⁴ is a hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₄ alkynyl or a cation selected from a Group 1 metal ion, a Group 2 metal ion, a primary ammonium ion or a secondary ammonium ion; and
R²² is a hydrogen atom or carboxy.

11. A compound of Formula 42, 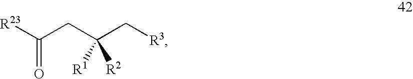
including complexes, salts, solvates, hydrates, opposite enantiomers, diastereomers, geometric isomers, and mixtures thereof, in which:
R¹ and R² are independently hydrogen atoms or C₁₋₃ alkyl optionally substituted with one to five fluorine atoms, provided that when R¹ is a hydrogen atom, R² is not a hydrogen atom;
R³ is C₁₋₆ alkyl, C₃₋₆ cycloalkyl, C₃₋₆ cycloalkyl-C₁₋₆ alkyl, aryl, aryl-C₁₋₃ alkyl, or arylamino, wherein each alkyl of R³ is optionally substituted with one to five fluorine atoms, and each aryl of R³ is optionally substituted with from one to three substituents independently selected from chloro, fluoro, amino, nitro, cyano, C₁₋₃ alkylamino, C₁₋₃ alkyl optionally substituted with one to three fluorine atoms, and C₁₋₃ alkoxy optionally substituted with from one to three fluorine atoms; and
R²³ is a hydrogen atom or a chiral oxazolidin-2-one-3-yl.

12. A compound selected from:
(R)-5-methyl-3-oxo-heptanoic acid ethyl ester;
(R)-5-methyl-3-oxo-octanoic acid ethyl ester;
(R)-5-methyl-3-oxo-nonanoic acid ethyl ester;
(R,Z)-3-amino-5-methyl-hept-2-enoic acid ethyl ester;
(R,Z)-3-amino-5-methyl-oct-2-enoic acid ethyl ester;
(R,Z)-3-amino-5-methyl-non-2-enoic acid ethyl ester;
(R,Z)-3-acetylamino-5-methyl-hept-2-enoic acid ethyl ester;
(R,Z)-3-acetylamino-5-methyl-oct-2-enoic acid ethyl ester;
(R,Z)-3-acetylamino-5-methyl-non-2-enoic acid ethyl ester;
(3S,5R)-3-amino-5-methyl-heptanoic acid ethyl ester;
(3S,5R)-3-amino-5-methyl-octanoic acid ethyl ester;
(3S,5R)-3-amino-5-methyl-nonanoic acid ethyl ester;
(3S,5R)-3-acetylamino-5-methyl-heptanoic acid ethyl ester;
(3S,5R)-3-acetylamino-5-methyl-octanoic acid ethyl ester;
(3S,5R)-3-acetylamino-5-methyl-nonanoic acid ethyl ester;
(3S,5R)-3-acetylamino-5-methyl-heptanoic acid;
(3S,5R)-3-acetylamino-5-methyl-octanoic acid;
(3S,5R)-3-acetylamino-5-methyl-nonanoic acid;
(3R,5R)-3-hydroxy-5-methyl-heptanoic acid;
(3R,5R)-3-hydroxy-5-methyl-octanoic acid;
(3R,5R)-3-hydroxy-5-methyl-nonanoic acid;
(3R,5R)-3-hydroxy-5-methyl-heptanoic acid benzyloxy-amide;
(3R,5R)-3-hydroxy-5-methyl-octanoic acid benzyloxy-amide;
(3R,5R)-3-hydroxy-5-methyl-nonanoic acid benzyloxy-amide;
(3R,5R)-3-hydroxy-5-methyl-heptanoic acid ethyl ester;
(3R,5R)-3-hydroxy-5-methyl-octanoic acid ethyl ester;
(3R,5R)-3-hydroxy-5-methyl-nonanoic acid ethyl ester;
(2R,4S)-1-benzyloxy-4-(2-methyl-butyl)-azetidin-2-one;
(2R,4S)-1-benzyloxy-4-(2-methyl-pentyl)-azetidin-2-one;
(2R,4S)-1-benzyloxy-4-(2-methyl-hexyl)-azetidin-2-one;
(3S,5R)-3-benzyloxyamino-5-methyl-heptanoic acid;
(3S,5R)-3-benzyloxyamino-5-methyl-octanoic acid;
(3S,5R)-3-benzyloxyamino-5-methyl-nonanoic acid;
(1S,3S,5R)-3-[benzyl-(1-phenyl-ethyl)-amino]-5-methyl-heptanoic acid ethyl ester;
(1S,3S,5R)-3-[benzyl-(1-phenyl-ethyl)-amino]-5-methyl-octanoic acid ethyl ester;
(1S,3S,5R)-3-[benzyl-(1-phenyl-ethyl)-amino]-5-methyl-nonanoic acid ethyl ester;
(5R)-3-hydroxy-5-methyl-heptanoic acid ethyl ester;
(5R)-3-hydroxy-5-methyl-octanoic acid ethyl ester;
(5R)-3-hydroxy-5-methyl-nonanoic acid ethyl ester;
(R,E)-5-methyl-hept-2-enoic acid ethyl ester;
(R,E)-5-methyl-oct-2-enoic acid ethyl ester;
(R,E)-5-methyl-non-2-enoic acid ethyl ester;
(R,E)-5-methyl-hept-3-enoic acid ethyl ester;
(R,E)-5-methyl-oct-3-enoic acid ethyl ester;
(R,E)-5-methyl-non-3-enoic acid ethyl ester;
(5R)-5-methyl-3-(toluene-4-sulfonyloxy)-heptanoic acid ethyl ester;
(5R)-5-methyl-3-(toluene-4-sulfonyloxy)-octanoic acid ethyl ester;
(5R)-5-methyl-3-(toluene-4-sulfonyloxy)-nonanoic acid ethyl ester;
(5R)-3-methanesulfonyloxy-5-methyl-heptanoic acid ethyl ester;
(5R)-3-methanesulfonyloxy-5-methyl-octanoic acid ethyl ester;
(R)-3-methanesulfonyloxy-5-methyl-nonanoic acid ethyl ester;
(R)-1-imidazol-1-yl-3-methyl-pentan-1-one;
(R)-1-imidazol-1-yl-3-methyl-hexan-1-one;
(R)-1-imidazol-1-yl-3-methyl-heptan-1-one; and
the pharmaceutically acceptable complexes, salts, solvates, hydrates, opposite enantiomers, diastereomers, geometric isomers, and mixtures thereof.
